(19) United States
(12) Patent Application Publication
(10) Pub. No.: US 2002/0187936 A1
(43) Pub. Date: Dec. 12, 2002

(54) Methods of treating liver disease and liver damage with growth hormone and foxM1B

(75) Inventors: Robert H. Costa, Oak Park, IL (US); Xinhe Wang, Chicago, IL (US); Guy Adami, Brookfield, IL (US); Yongjun Tan, Arlington Heights, IL (US); Katherine Krupczak-Hollis, Chicago, IL (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Board of Trustees for the University of Illinois

(21) Appl. No.: 10/151,587

(22) Filed: May 17, 2002

Related U.S. Application Data
(60) Provisional application No. 60/291,789, filed on May 17, 2001.
(60) Provisional application No. 60/305,821, filed on Jul. 16, 2001.
(60) Provisional application No. 60/315,484, filed on Aug. 28, 2001.

(51) Int. Cl. A61K 38/18; A61K 48/00; C12N 5/08; C12N 15/86

(52) U.S. Cl. 514/12; 514/44; 435/455; 435/370; 424/93.2; 435/456

(57) ABSTRACT

The invention provides a method of treating liver damage or disease in a patient by stimulating liver regeneration. Specifically, the invention provides a method of inducing liver cell proliferation comprising contacting liver cells that express FoxM1B protein with growth hormone. The invention also provides methods of screening for compounds that induce FoxM1B protein expression, nuclear localization, or both expression and nuclear localization. The invention further provides pharmaceutical compositions comprising selected compounds and methods of using such compositions.

This application is related to U.S. provisional application Serial No. 60/291,789, filed May 17, 2001, No. 60/305,821, filed Jul. 16, 2001, and No. 60/315,484, filed Aug. 28, 2001.

This application was supported by a Public Service grant from the National Institutes of Diabetes and Digestive and Kidney Diseases, grant number DK54687. The U.S. government may have certain rights to this invention.

### BACKGROUND OF THE INVENTION

1. Field of the Invention

This invention relates to methods of treating liver diseases and liver damage by inducing expression and nuclear localization of FoxM1B protein. The invention particularly relates to methods of inducing FoxM1B protein expression and inducing or facilitating translocation of FoxM1B protein to the nucleus of a mammalian cell, where it potentiates transcription of many essential cell cycle promotion genes. Specifically, the invention relates to methods of preventing or ameliorating liver damage or disease comprising administering to a patient a therapeutically effective amount of growth hormone. The invention further relates to methods of screening compounds that induce expression of FoxM1B, induce nuclear localization of FoxM1B, or induce both expression and nuclear localization of FoxM1B protein in liver cells. The invention also provides such compounds that are useful for preventing or ameliorating liver damage or disease, and methods for using said compounds for preventing or ameliorating liver damage or disease. 2. Background of the Related Art

One important function of mammalian liver is to detoxify harmful compounds that enter the body. In the liver, toxic substances may be cleared from the body by phagocytosis, secretion into the bile, or by chemical modification of the compound to facilitate elimination by the kidneys. Other functions of the liver include storing vitamins, producing cholesterol and bile to assist digestion, converting excess glucose into glycogen, and releasing glucose into the blood during fasting. The liver is also responsible for secreting all serum carrier proteins and proteins involved in blood coagulation. A healthy liver, therefore, is an important contributor to the overall health of an animal or human individual.

Environmental and dietary toxins constantly bombard the liver throughout a lifetime. The potential for liver damage increases with time and as the stress of removing these toxins increases. The mammalian liver is capable of completely regenerating itself in response to such liver damage (Fausto et al., 1995, FASEB J 9: 1527-1536; Michalopoulos et al., 1997, Science 276: 60-66; Taub, 1996, FASEB J. 10: 413-427). However, excessive exposure to toxins such as alcohol or certain drugs can cause severe liver damage leading to disease. During aging, the ability of the liver to regenerate decreases and liver damage and disease becomes more severe and more difficult to treat. Thus, the ability to stimulate hepatocyte proliferation and restore the regenerative potential of these liver cells would prove invaluable in treating liver diseases.

During the aging process, the expression patterns of several genes involved in regulating the cell cycle become altered. These defects in the mitotic machinery contribute to chromosome instability and mutations that lead to many diseases found in the elderly (Ly et al., 2000, Science 287: 2486-2492). Diminished expression of several cell cycle regulatory genes, in particular the Forkhead Box M1B (FoxM1B) transcription factor (also known as Trident and HFH-11B) contribute to age-related defects in cellular proliferation (Id). FoxM1B is a proliferation-specific transcription factor that shares 39% amino acid homology with the HNF-3 winged helix DNA binding domain. The molecule also contains a potent C-terminal transcriptional activation domain that possesses several phosphorylation sites for M-phase specific kinases as well as PEST sequences that mediate rapid protein degradation (Korver et al., 1997, Nucleic Acids Res. 25:1715-1719; Korver et al., 1997, Genomics 46:435-442; Yao et al, 1997, J Biol. Chem. 272:19827-19836; Ye et al., 1997, Mol. Cell Biol. 17:1626-1641).

FoxM1B is expressed in several tumor-derived epithelial cell lines and is induced by serum prior to the G₁/S transition (Korver et al., 1997, Nucleic Acids Res. 25: 1715-1719; Korver et al., 1997, Genomics 46:435-442; Yao et al., 1997, J Biol. Chem. 272: 19827-19836; Ye et al., 1997, Mol. Cell Biol. 17: 1626-1641). In situ hybridization studies show that FoxM1B is expressed in embryonic liver, intestine, lung, and renal pelvis (Ye et al., 1997, Mol. Cell Biol. 17: 1626-1641). In adult tissue, however, FoxM1B is not expressed in postmitotic, differentiated cells of the liver and lung, although it is expressed in proliferating cells of the thymus, testis, small intestine, and colon (Id). FoxM1B expression is reactivated in the liver prior to hepatocyte DNA replication following regeneration induced by partial hepatectomy (Id).

Liver regeneration studies with transgenic mice expressing a transcriptionally active FoxM1B gene in hepatocytes demonstrated that early expression of FoxM1B advanced the onset of hepatocyte DNA replication and mitosis by 8 hours (Ye et al., 1999, Mol. Cell Biol. 19: 8570-8580). Abnormal hepatocyte proliferation in nonregenerating livers in transgenic mice was not observed, and this was found to be because FoxM1B was retained in the cytoplasm rather than being translocated to the nucleus (Id). FoxM1B was found to be translocated to the nucleus only in response to mitogenic signaling during liver regeneration (Id). Analyzing RNA from wild type and transgenic regenerating livers by differential hybridization of cDNA array blots and RNase protection assays showed that FoxM1B stimulated the expression of several cell cycle regulatory genes (Id). The data show that FoxM1B either directly or indirectly mediates cell cycle progression.

Expression of the c-myc transcription factor and tumor growth factor α (TGF-α) in transgenic mouse hepatocytes can also stimulate hepatocyte replication during liver regeneration. However, constitutive expression of c-myc or TGF-α increases the incidence of liver tumors (Factor et al., 1997, Hepatology 26: 1434-1443). Co-expression of c-myc and TGF-α in hepatocytes also stimulates oxidative stress and DNA damage leading to senescence after partial hepatectomy and the development of liver tumors between 4 to 8 months of age (Id; Factor et al., 1998, J Biol. Chem. 273: 15846-15853).

While FoxM1B can potentiate transcription of cell cycle promotion genes and thus stimulate hepatocyte replication that can offset toxin- and age-associated liver damage, it does so only when translocated into the nucleus (Ye et al., 1999, Mol. Cell Biol. 19: 8570-8580). Expression of FoxM1B in adult liver followed by its induction to enter the nucleus at the appropriate time may alleviate age-related proliferation defects and avoid unwanted hepatocyte proliferation, making it a far safer candidate for therapeutic intervention than compounds that induce expression of c-myc or TGF-α.

### SUMMARY OF THE INVENTION

This invention provides methods of restoring hepatocyte DNA replication and mitosis in diseased and damaged livers. The invention also provides methods of inducing expression and nuclear localization of FoxM1B protein in mammalian liver cells, particularly aged or toxin-damaged liver cells. In one aspect, the invention provides methods for inducing expression, nuclear localization or expression and nuclear localization of FoxM1B protein by contacting liver cells with growth hormone. In another aspect, the invention provides screening methods to identify compounds having the ability to induce expression of FoxM1B protein, compounds that induce nuclear localization of FoxM1B protein, and compounds that induce both expression and nuclear localization of FoxM1B protein in mammalian cells. In a further aspect, the invention provides pharmaceutical compositions comprising compounds identified by the screening methods of the invention. In yet a further aspect, the invention provides methods of preventing or ameliorating liver damage in patients in need of such treatment.

In a particular aspect, the invention provides methods for inducing nuclear localization of FoxM1B protein in a mammalian liver cell comprising the step of contacting the liver cell with growth hormone. In one embodiment, the mammalian liver cell expresses FoxM1B endogenously, such as in liver cells from a young mammal. In another embodiment, the liver cells have a reduced ability to express FoxM1B protein, such as liver cells in aged mammals. In a preferred embodiment, the invention provides a recombinant nucleic acid construct that can be introduced into a cell, preferably a liver cell and most preferably a hepatocyte cell to restore FoxM1B expression and regenerative potential in the cell.

In another aspect, the invention provides recombinant nucleic acid constructs that comprise nucleic acid having a nucleotide sequence encoding FoxM1B protein. In a preferred embodiment, the nucleic acid encodes human FoxM1B and has the nucleotide sequence that encodes a protein as set forth in SEQ ID NO: 2. The recombinant nucleic acid construct also comprises an expression control sequence that is operatively linked to the nucleic acid encoding FoxM1B. In one aspect, the expression control sequence is a liver-specific promoter that is specifically active in liver cells. In this embodiment, the nucleic acid comprising recombinant nucleic acid construct of the invention is transcriptionally active and expressed only in liver cells when the construct is delivered in vivo. Promoters useful in this aspect of the invention include, but are not limited to, human or mouse α1-antitrypsin promoter, albumin promoter, serum amyloid A promoter, transthyretin promoter, and hepatocyte nuclear factor 6 (HNF-6) promoter. Preferably, the promoter is HNF-6, which is induced by growth hormone.

In certain aspects, a recombinant nucleic acid construct of the invention comprises a vector. In particular embodiments, the vector is a viral vector, such as an adenovirus, an adeno-associated virus, a retrovirus, herpes simplex virus, or vaccinia virus vector.

The invention further provides methods for introducing the recombinant nucleic acid constructs of the invention into cells, most preferably mammalian cells. In a preferred embodiment, recombinant expression constructs of the invention are formulated into liposomes and introduced into mammalian liver cells. Other proliferative cell types that may benefit from FoxM1B intervention are, for example, intestinal and colonic epithelial cells, thymocytes in the thymus and lymphocytes in the spleen, and basal cells of the skin. Recombinant expression constructs of the invention can also be introduced into cells using, for example, the ExGen 500 reagent (MBI Fermentas).

The invention also provides cells, preferably mammalian cells, into which have been introduced a recombinant nucleic acid construct of the invention. In preferred embodiments, the cells are hepatocytes, intestinal or colonic epithelial cells, thymocytes in the thymus and lymphocytes in the spleen, or basal cells of the skin In another aspect, the invention provides methods of stimulating liver regeneration in cells that express FoxM1B protein by inducing FoxM1B protein to translocate into the nucleus of the cells. In a particular aspect, the invention provides a method for inducing nuclear localization contacting the cells with growth hormone.

The invention further provides a method of screening for compounds that induce expression of FoxM1B protein in mammalian cells, wherein the FoxM1B protein can be translocated into the nucleus. In these embodiments, the inventive methods comprise the steps of contacting a plurality of cells that do not express FoxM1B under conventional culture conditions, with a candidate compound in the presence and absence of growth hormone; assaying FoxM1B expression and localization in the cells cultured in the presence and absence of growth hormone and comparing FoxM1B expression and nuclear localization in the cells, wherein a candidate compound is identified when FoxM1B is expressed in said cells and localized in the nuclei of cells in the presence but not in the absence of growth hormone.

The invention also provides a method of screening for compounds that induce nuclear localization of FoxM1B protein. In these embodiments, the inventive methods comprise the steps of contacting cells that express FoxM1B protein with a candidate compound and examining the intracellular localization of FoxM1B protein in the cell; wherein the candidate compound is identified when FoxM1B protein is localized in the nucleus of the cell in the presence of the compound but not in the absence of the compound.

The invention also provides methods of screening for compounds that induce both expression and nuclear localization of FoxM1B protein. In these embodiments, the methods of the invention comprise the steps of (a) contacting cells that do not express FoxM1B under conventional culture conditions, with a candidate compound; and (b) assaying FoxM1B expression and localization in the cells, wherein a candidate compound is identified when FoxM1B is expressed and localized in the nuclei of cells contacted with the compound but not in cells not contacted with the compound. In alternative embodiments, the cells are contacted with growth hormone upon induction of FoxM1B expression in the cells in the presence of the compound.

The invention also provides methods of inducing liver cell proliferation comprising the step of contacting a liver cell with growth hormone or a compound identified in a screening method of the invention, wherein the liver cell expresses FoxM1B protein. In a preferred embodiment, the cell expresses FoxM1B protein endogenously, i.e., encoded by the cellular DNA. In another embodiment, the cell expresses FoxM1B encoded by a recombinant nucleic acid construct of the invention.

The invention further provides methods of stimulating liver regeneration in a mammal, comprising the step of contacting mammalian liver cells with growth hormone or a compound identified in a screening method of the invention. In a preferred embodiment, the cell expresses FoxM1B protein endogenously, i.e., encoded by the cellular DNA. In another embodiment, the cell expresses FoxM1B encoded by a recombinant nucleic acid construct of the invention.

The invention also provides methods of preventing or ameliorating liver damage in a mammal comprising the step of contacting mammalian liver cells with growth hormone or a compound identified in a screening method of the invention. In a preferred embodiment, the cell expresses FoxM1B protein endogenously, i.e., encoded by the cellular DNA. In another embodiment, the cell expresses FoxM1B encoded by a recombinant nucleic acid construct of the invention. In a particular aspect, the method is a preventative measure, most preferably applied to individuals with a high susceptibility or a genetic disposition for acquiring liver damage or liver disease.

In another aspect, the method is a therapeutic measure, applied to an individual who suffers from liver damage or liver disease. In this aspect, the methods of the invention prevent further damage or disease progression or reverses damage or disease progression. In a preferred embodiment, the methods are applied to an individual awaiting a liver transplant. In other preferred embodiments, the methods of the invention are applied to a liver removed from a donor to be transplanted into a recipient. In one embodiment, the donor is treated with growth hormone or another compound identified in a screening method of the invention prior to surgical removal of the liver to induce expression, nuclear localization or expression and nuclear localization of FoxM1B protein. In another aspect, the liver is contacted with growth hormone or another compound identified in a screening method of the invention that induces expression, nuclear localization or expression and nuclear localization of FoxM1B protein after removal from the donor. The methods of the invention can also be applied to the recipient, by treating the recipient with growth hormone or another compound identified in a screening method of the invention that induces expression, nuclear localization or expression and nuclear localization of FoxM1B protein after the liver has been transplanted.

The invention further provides methods of preventing or ameliorating liver damage in a mammal comprising the steps of introducing into the mammal liver cells that express FoxM1B protein and thereafter contacting the liver cells with growth hormone or another compound identified in a screening method of the invention. In this aspect, liver cells are removed from an individual and reintroduced into a recipient individual, most preferably the same individual to minimize immunological complications. In preferred embodiments, the liver cells express FoxM1B endogenously. In another preferred embodiment, the liver cells are contacted ex vivo with a recombinant nucleic acid construct of the invention whereby the cells express FoxM1B protein. Both allografts and autografts as disclosed herein are contemplated by the invention to protect or ameliorate liver damage or liver disease in a patient. The invention provides these methods wherein the liver cells removed from an individual are contacted with growth hormone or a compound identified in a screening method of the invention that induces expression, nuclear localization or expression and nuclear localization of FoxM1B protein prior to or after introducing the cells into a recipient.

Diminished expression of FoxM1B and its target genes mediating cell cycle regulation is associated with reduced proliferation in regenerating hepatocytes of 12-month old (old-aged) mice and in proliferating fibroblast of old-aged humans. Liver regeneration studies disclosed herein using old-aged TTR-FoxM1B transgenic mice demonstrate that maintaining FoxM1B levels restores hepatocyte proliferation and expression of genes that regulate cell division. Acute delivery of FoxM1B protein to old aged mice using Adenovirus gene therapy restores hepatocyte DNA replication and cell division during liver regeneration. These data suggests that FoxM1B gene delivery is advantageous for therapeutic intervention to restore proliferation due to diminished FoxM1B levels. This use is supported by results disclosed herein using genetically altered mice in which hepatocytes are deficient in the FoxM1B gene. FoxM1B deficiency inhibits hepatocyte proliferation even in young mice in response to liver injury, demonstrating that FoxM1B expression is essential for hepatocytes to undergo DNA replication and cell division (mitosis). FoxM1B is also essential for hepatocyte replication required to regenerate the liver in response to injury. Because FoxM1B is expressed in every proliferating cell that has been examined, FoxM1B is critical for proliferation of all cell types in the body. Taken together, the instant disclosure demonstrates that expression of FoxM1B is necessary for hepatocyte replication in response to liver injury and that increased FoxM1B levels is sufficient to restore hepatocyte proliferation in the elderly and in patients with liver diseases. Thus, the methods disclosed herein provide advantages for treating and preventing liver disease and injury.

Further, the methods disclosed herein have important advantages over other methods known in the art for inducing hepatocyte proliferation. An example is hepatocyte expression of the c-myc in transgenic mice, which stimulates hepatocyte replication during liver regeneration. Constitutive c-myc expression is undesirable because it causes aberrant hepatocyte proliferation in the absence of liver injury. This is due to c-myc localizing to the nucleus in the absence of proliferative signals, and results in development of liver cancer such as hepatocellular carcinoma. Unlike c-myc, FoxM1B nuclear localization requires proliferation-specific signals. Therefore, ectopic FoxM1B expression is insufficient to induce quiescent cells to enter the cell cycle, and thus will not induce unwanted cellular proliferation. This feature permits FoxM1B to be used for therapeutic intervention to ameliorate defective proliferation observed in the elderly population or patients with liver diseases exhibiting defective liver regeneration, without implicating the risk of the patients developing liver cancers such as hepatocellular carcinoma. Because increased FoxM1B expression in quiescent cells does not induce unwanted cellular proliferation leading to the development of cancer, it is much safer for administration to patients to stimulate liver regeneration.

Specific preferred embodiments of the invention will become evident from the following more detailed description of certain preferred embodiments and the claims.

### DETAILED DESCRIPTION OF THE DRAWINGS

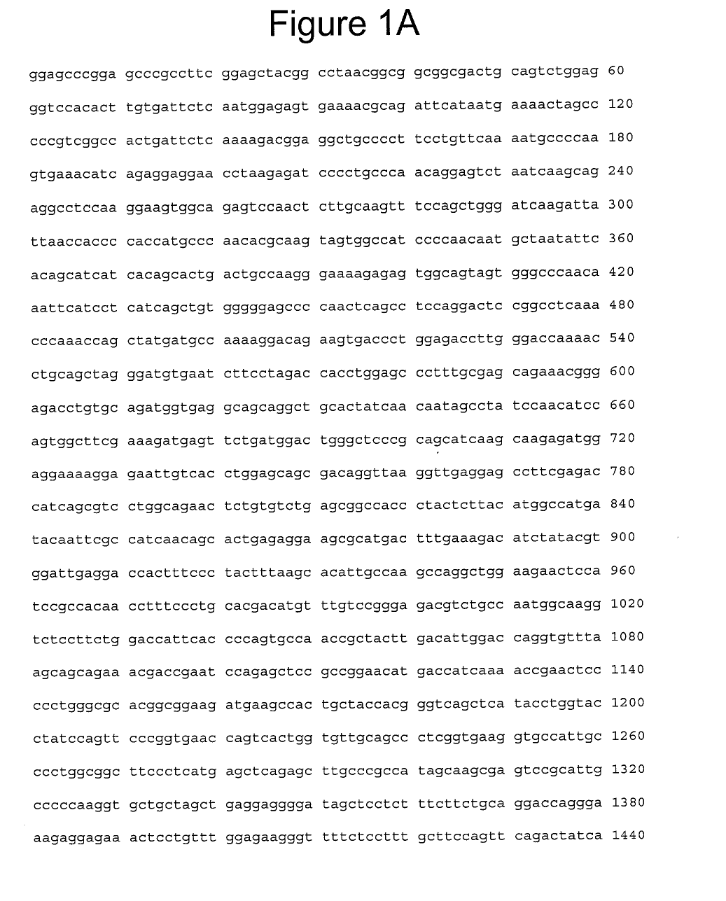

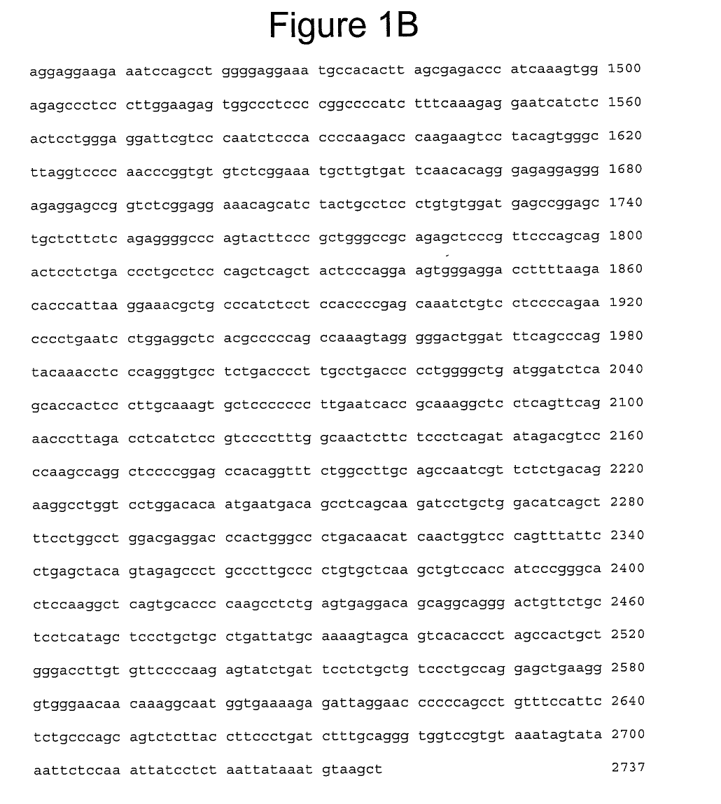

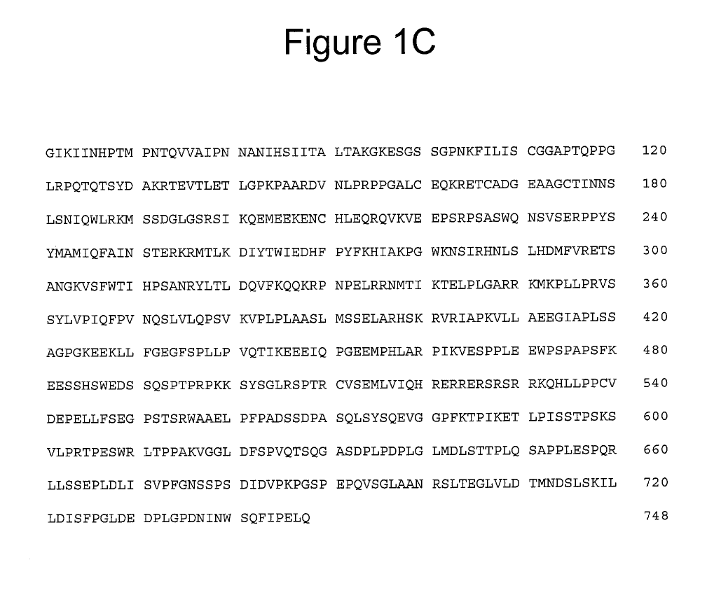

FIGS. 1A-B depicts human FoxM1B cDNA comprising a deletion of the terminal 972 nucleotides at the 3′ end (SEQ ID NO: 1).

FIG. 1C depicts human FoxM1B protein sequence (SEQ ID NO: 2) encoded by the nucleotide sequence as set forth in SEQ ID NO: 1.

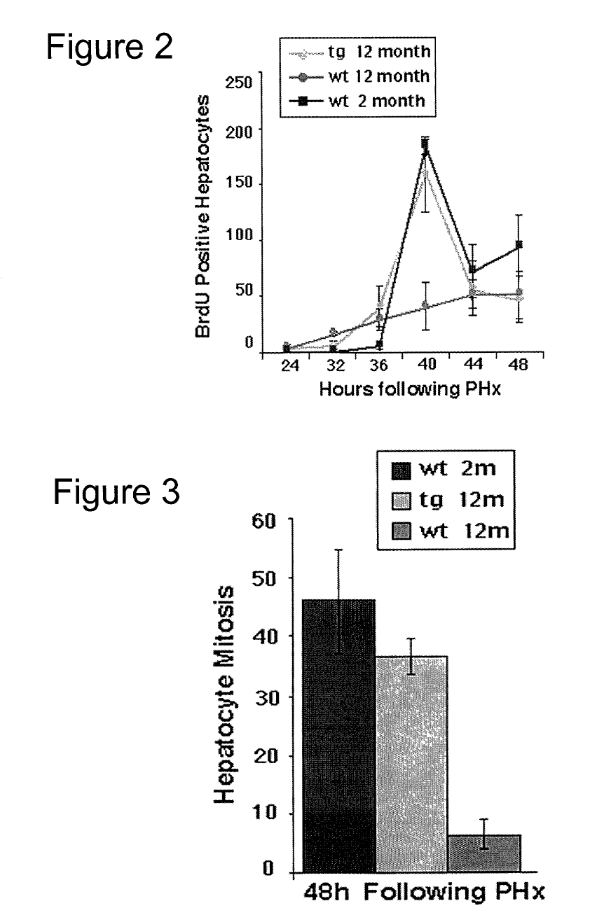

FIG. 2 shows a graph representing 5-bromo-2′-deoxy-uridine (BrdU) incorporation (as a measure of DNA replication) at the indicated hours after partial hepatectomy (PHx) in twelve month old wild type CD-1 mice (WT, solid circles), twelve month old transgenic CD-1 mice (TG, solid diamonds), or two month old wild type CD-1 mice (solid squares).

FIG. 3 shows a graph representing increased hepatocyte mitosis in regenerating livers of old-aged TG mice at 48 hours post PHx.

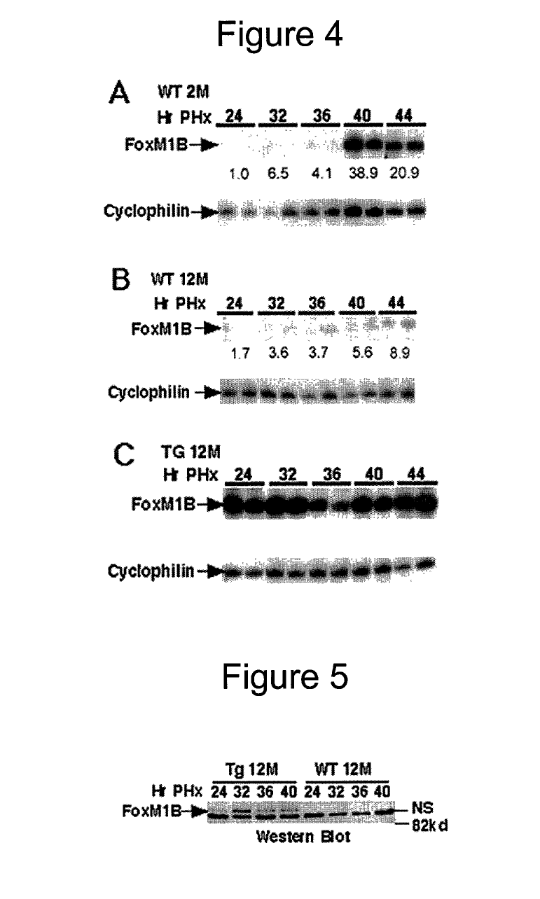

FIG. 4 shows RNase protection assays performed using total RNA isolated at the indicated hours post PHx from regenerating liver of two-month-old WT mice (A), twelve-month-old WT mice (B), and twelve month old TG mice (C).

FIG. 5 shows a western blot analysis with anti-FoxM1B antibodies performed with total liver protein extracts isolated from regenerating livers of twelve month old WT and TG mice at the indicated time points. FoxM1B protein migrates more slowly than a non-specific (NS) band.

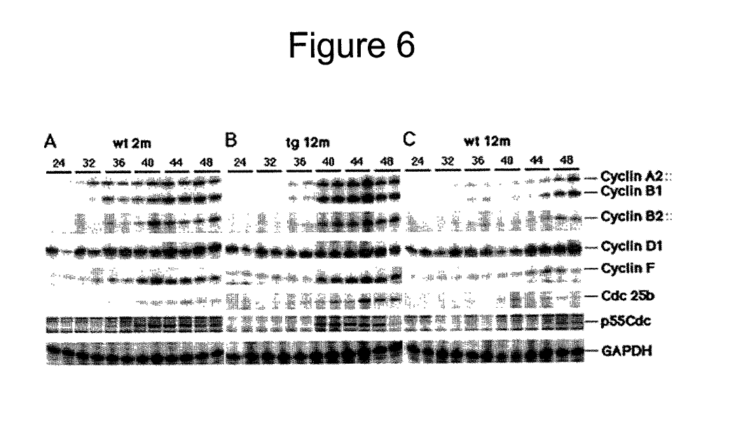

FIG. 6 shows an RNase protection assay demonstrating increased expression of cell cycle promotion genes in regenerating liver of old TG mice compared with WT mice at the indicated hours following PHx.

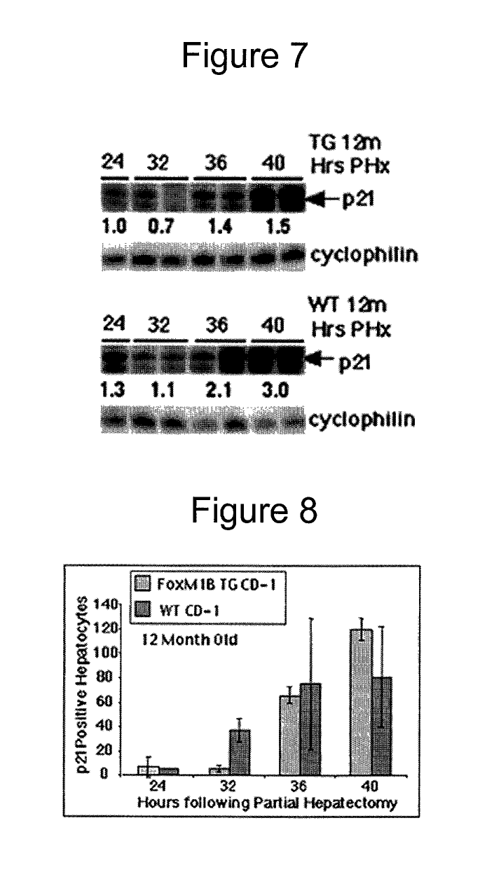

FIG. 7 shows an RNase protection assay of total RNA isolated from regenerating livers of twelve-month-old WT or TG mice using an antisense RNA probe for p21.

FIG. 8 shows a graph representing the number of p21 positive nuclei per 2500 hepatocytes per regenerating mouse liver, ±the standard deviation (SD).

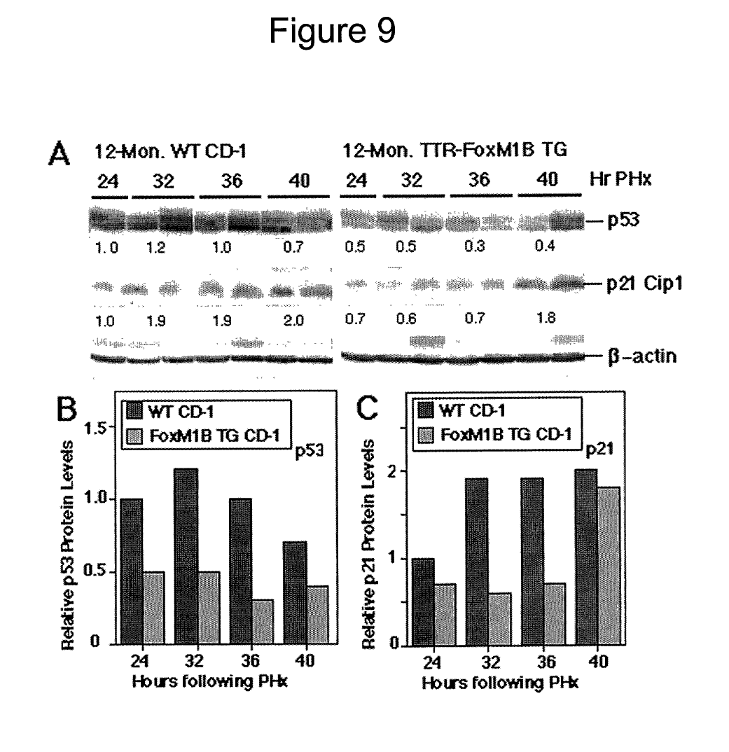

FIG. 9A depicts a Western blot with anti-p53 antibodies showing p53 protein expression in regenerating livers of old-aged TTR-FoxM1B TG mice and old-aged WT mice.

FIGS. 9B-C show graphs depicting relative p53 and p21 protein levels in old aged TTR-FoxM1B transgenic mice compared to levels in old-aged WT mice at various times after PHx.

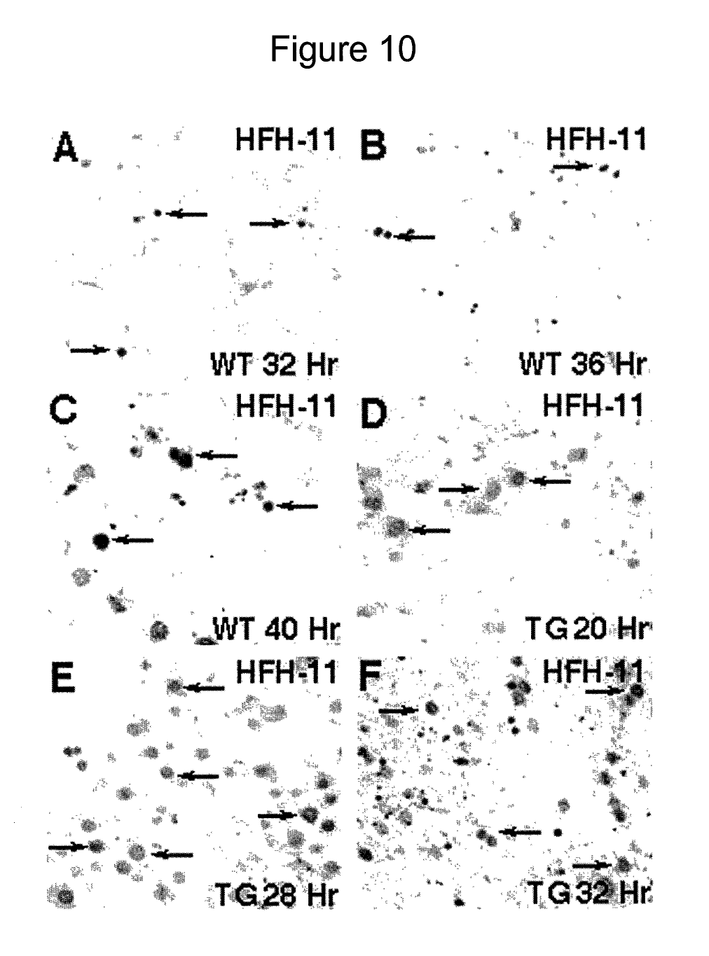

FIG. 10 shows immunohistochemical staining of FoxM1B protein with FoxM1B antibody and nuclear expression of FoxM1B protein in CCl₄-treated regenerating liver from WT (A-C) or TG (D-F) mice.

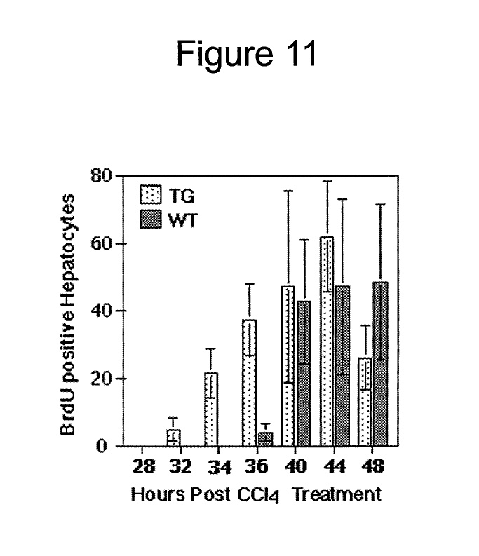

FIG. 11 shows a graph representing BrdU incorporation in hepatocytes at various time points after CCl₄-induced liver damage in WT and TG mice. BrdU positive cells were counted in three viewing fields, each field containing about 250 nuclei.

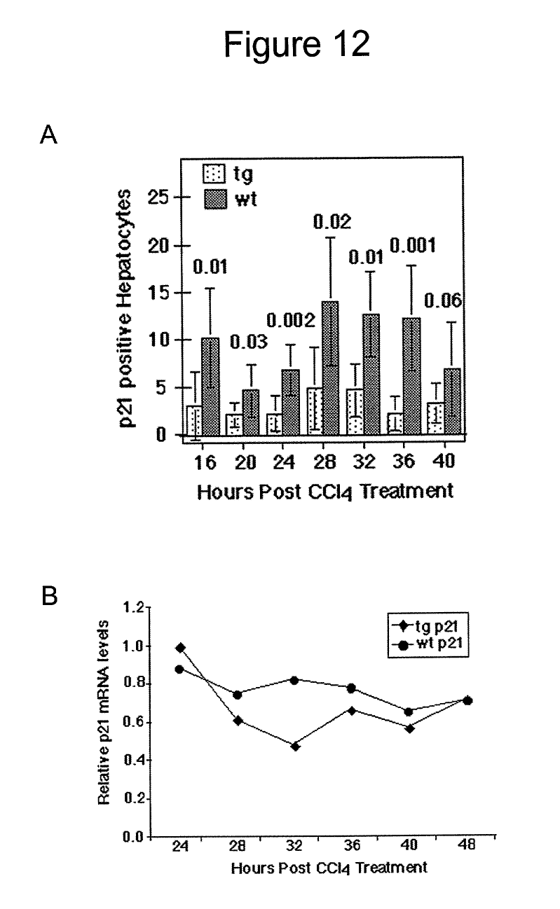

FIG. 12A shows a statistical analysis of p21-staining hepatocytes in WT and TG liver regeneration.

FIG. 12B shows a graph representing levels of p21 mRNA expression in regenerating livers from WT and TG mice, normalized to glyceraldehyde-3-phosphate dehydrogenase (GAPDH) and large ribosomal L32 protein levels.

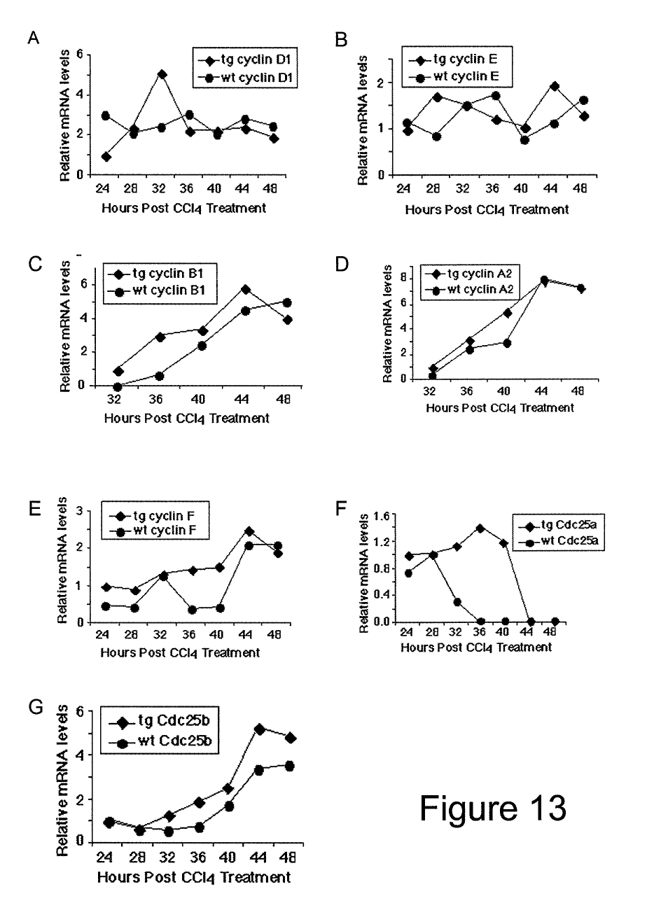

FIG. 13 shows a graph representing Cyclin D1 (A), Cyclin E (B), Cyclin B1 (C), Cyclin A2 (D), Cyclin F (E), Cdc25a (F), and Cdc25b (G) mRNA expression in regenerating WT and TG livers at various times after CCl₄ induced liver damage.

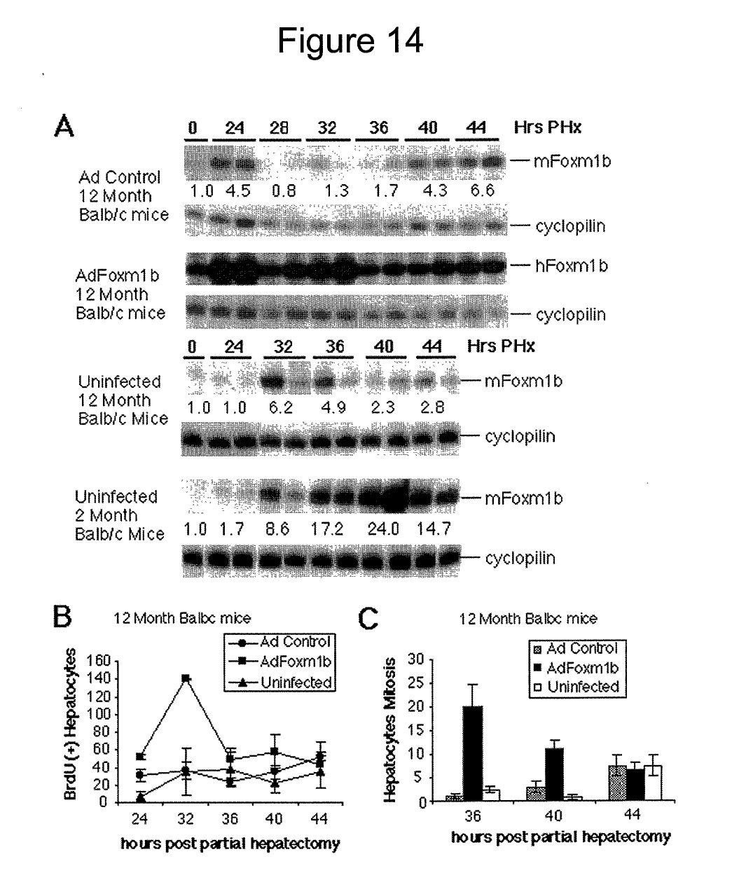

FIG. 14A shows FoxM1B mRNA levels in regenerating livers of old Balb/c mice infected with either AdCon (adenovirus control) or AdFoxM1B (adenoviral vector with FoxM1B) two days prior to PHx operation or left uninfected. Expression of FoxM1B mRNA was normalized to cyclophilin levels. Shown below the panel is the fold induction compared to expression levels at the beginning of the experiment (the 0-hour time point).

FIG. 14B shows a graph representing hepatocyte BrdU incorporation during mouse liver regeneration induced by PHx in twelve month-old Balb/c mice infected with either AdFoxM1B or AdCon or left uninfected. The mean of the number of BrdU positive nuclei per 1000 hepatocytes and the standard deviation (SD) was calculated for each time point.

FIG. 14C shows a graph representing increased hepatocyte mitosis in regenerating livers of old mice infected with AdFoxM1B between 36 to 44 hours post PHx. Using two regenerating livers for each time point post PHx, hepatocyte mitosis is expressed as the mean of the number of mitotic figures found per 1000 hepatocytes ±SD.

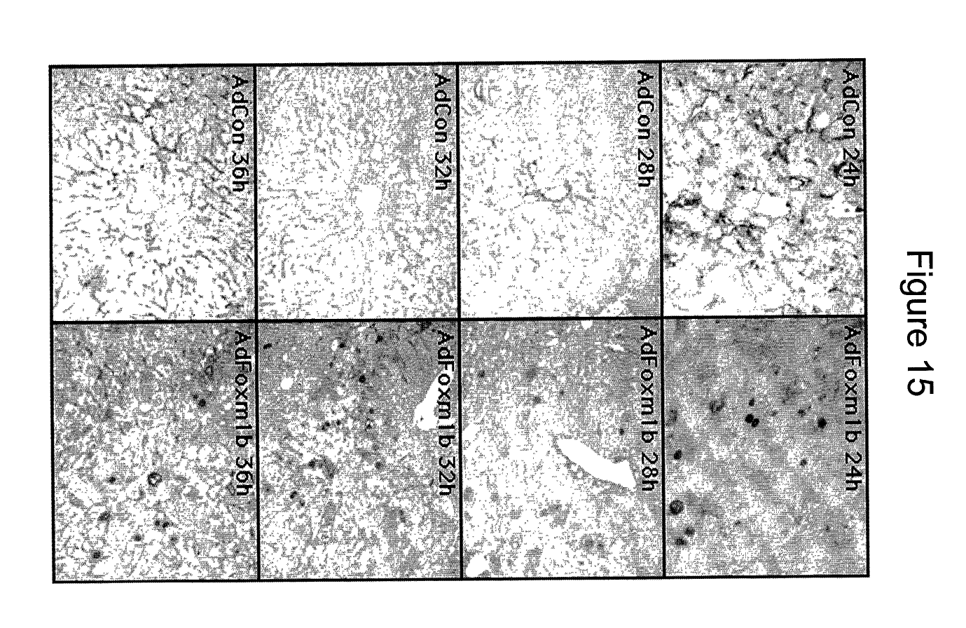

FIG. 15 shows immunohistochemical staining with FoxM1B antibody showing hepatocyte nuclear expression of FoxM1B protein in regenerating liver of old mice infected with AdFoxM1B but not with AdCon.

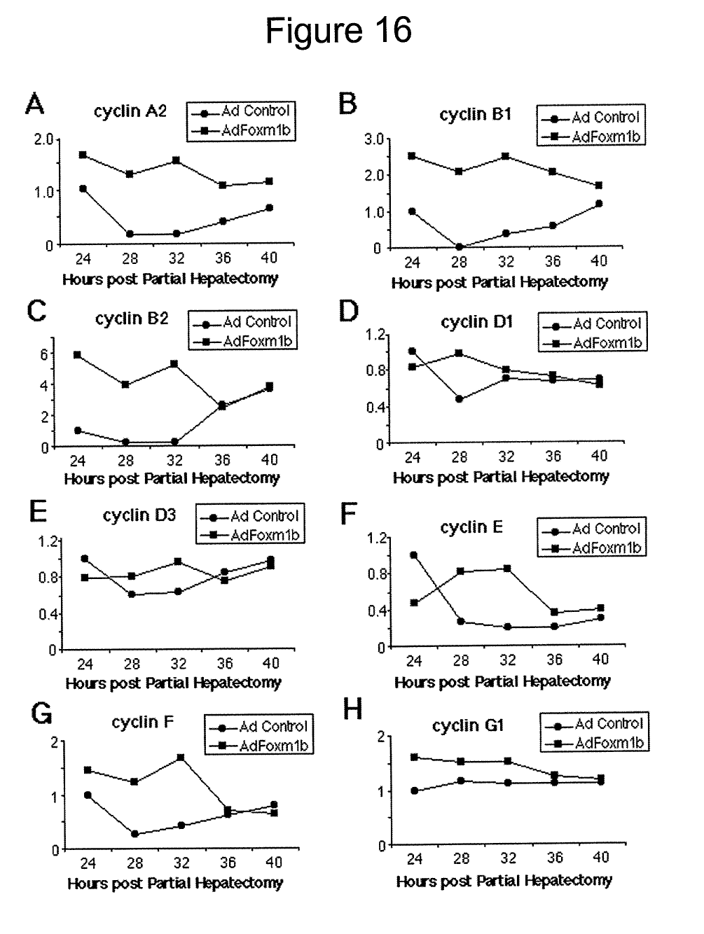

FIG. 16 shows a graph representing stimulated expression of cyclin genes in regenerating liver of old mice infected with AdFoxM1B. Expression levels of cyclin expression levels were normalized to the GAPDH and ribosome large subunit L32 protein mRNA levels. Graphic presentation of normalized mean mRNA levels of Cyclin A2 (A), Cyclin B1 (B), Cyclin B1 (C), Cyclin D1 (D), Cyclin D3 (E), Cyclin E (F), Cyclin F (G), and Cyclin G1 (H).

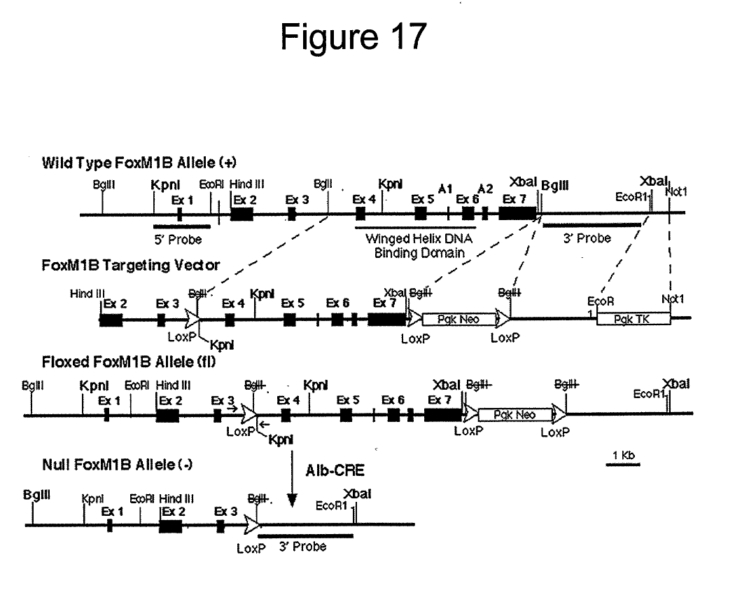

FIG. 17 is a schematic representation of triple-LoxP FoxM1B targeting vector used to generate the conditional FoxM1B knockout mice.

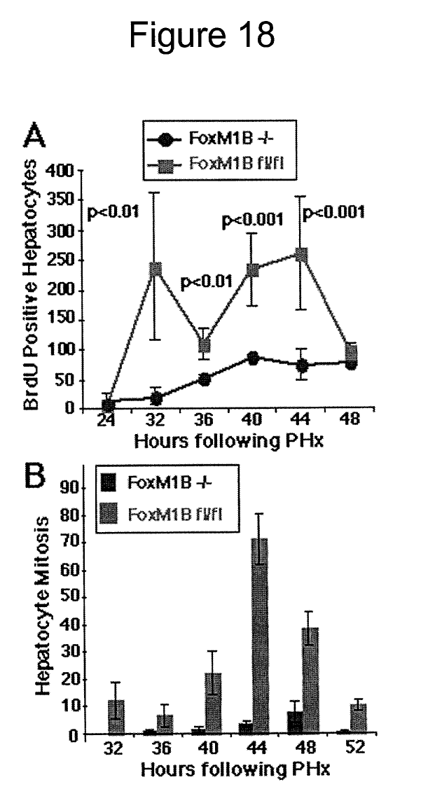

FIG. 18A depicts a graph showing BrdU incorporation in FoxM1B deficient hepatocytes after partial hepatectomy.

FIG. 18B depicts a graph showing hepatocyte mitosis at various time points after partial hepatectomy in FoxM1B−/− and FoxM1B fl/fl mice.

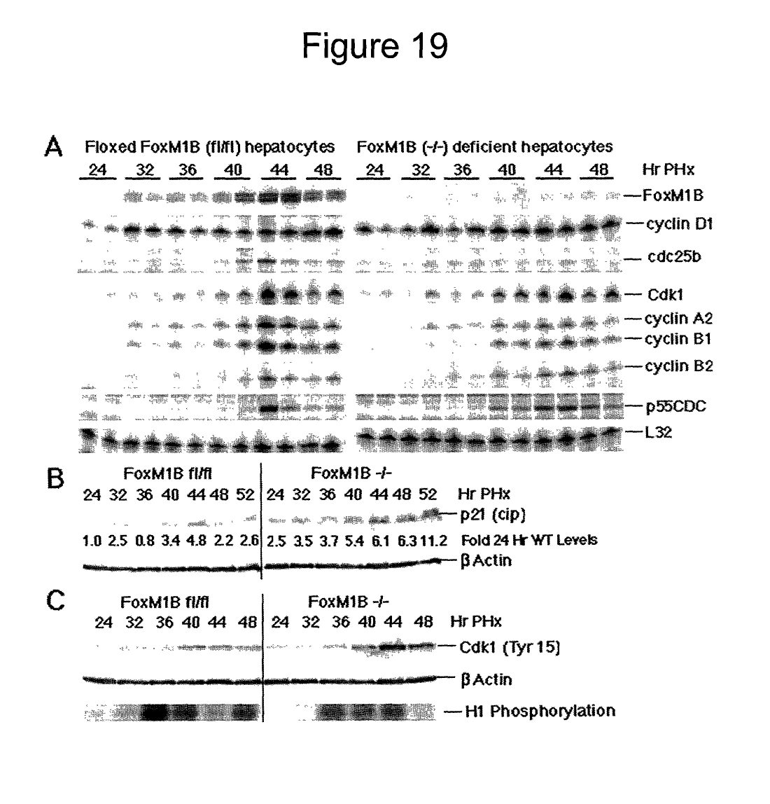

FIG. 19A depicts RNase protection assays performed in duplicate showing expression of cell cycle regulatory genes in regenerating liver of FoxM1B −/− and FoxM1B fl/fl mice.

FIG. 19B depicts a Western blot analysis showing p21 protein levels in regenerating FoxM1B −/− and FoxM1B fl/fl hepatocytes.

FIG. 19C depicts a Western blot analysis with cdk-1 specific phospho-Tyrosine antibodies and kinase assays using H1 protein as a substrate in FoxM1B −/− and FoxM1B fl/fl hepatocytes during liver regeneration.

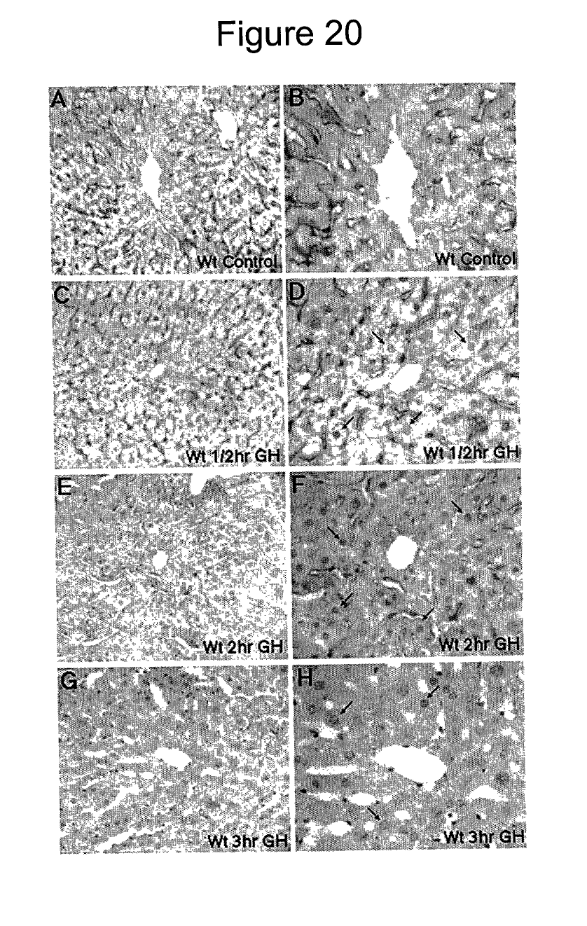

FIG. 20 shows hepatocyte nuclear expression of FoxM1B protein in young CD-1 mice stimulated by growth hormone. Shown are micrographs (200×, left panel and 400×, right panel) of wild-type liver sections displayed FoxM1B nuclear staining (indicated by arrows) between 30 minutes (C-D), 2 hours (E-F) and 3 hours (G-H) following growth hormone administration but not in control mice (A-B).

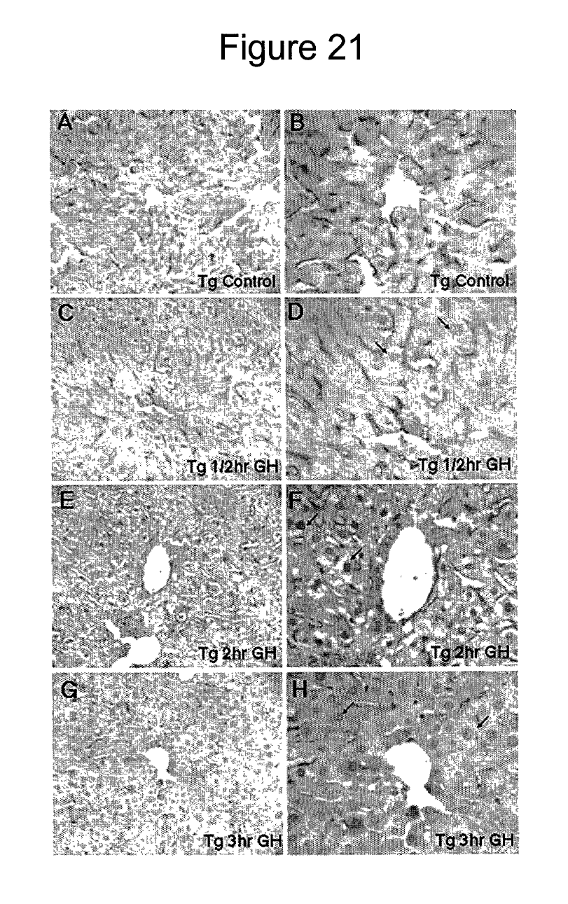

FIG. 21 shows hepatocyte nuclear expression of FoxM1B protein in young TTR-FoxM1B transgenic mice stimulated by growth hormone. Shown are micrographs (200×, left panel and 400×, right panel) of TTR-FoxM1B liver sections displayed FoxM1B nuclear staining (indicated by arrows) between 30 minutes (C-D), 2 hours (E-F) and 3 hours (G-H) following growth hormone administration but not in control transgenic mice (A-B).

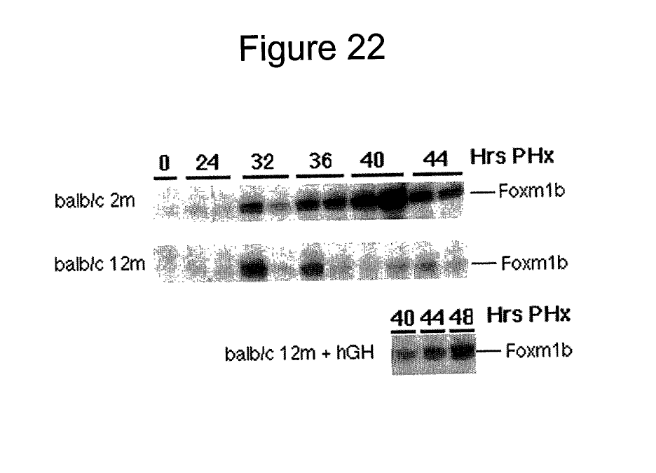

FIG. 22 shows a time course of FoxM1B mRNA levels in regenerating liver of untreated 2-month old (young) and 12-month old Balb/c mice as well as 12-month old Balb/c mice treated with human growth hormone.

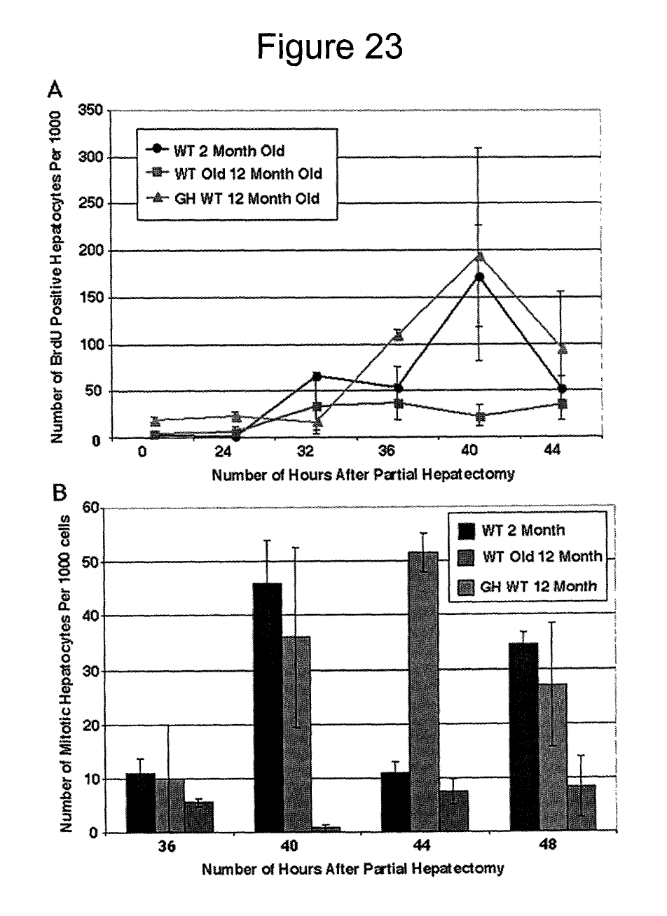

FIG. 23A shows a graph representing number of BrdU positive hepatocytes from regenerating livers in mice treated with growth hormone.

FIG. 23B shows a graph representing number of mitotic hepatocytes from regenerating livers in mice treated with growth hormone.

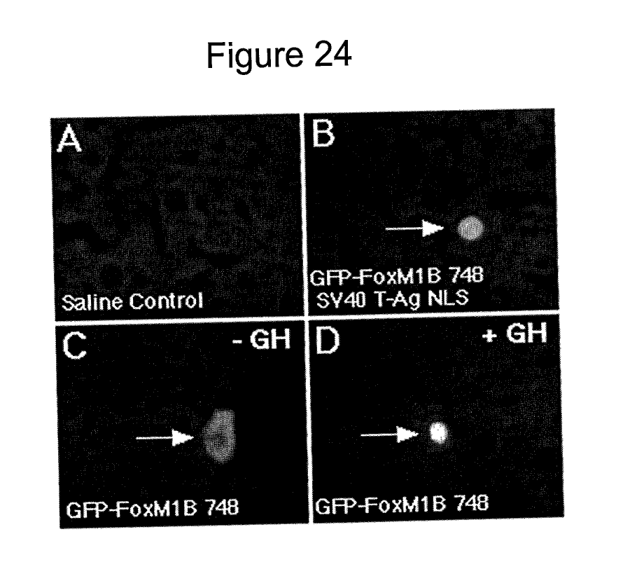

FIGS. 24A-D depicts immunohistochemical staining with FoxM1B antibody showing localization of GFP-FoxM1B-NLS (B) and GFP-FoxM1B in the presence and absence of growth hormone (C and D). Panel A is a control.

### DETAILED DESCRIPTION OF THE PREFERRED EMBODIMENTS

Standard techniques were used for recombinant DNA, oligonucleotide synthesis, and tissue culture and transformation (e.g., electroporation, lipofection). Enzymatic reactions and purification techniques were performed according to manufacturers' specifications or as commonly accomplished in the art or as described herein. The techniques and procedures were generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

Unless otherwise required by context, singular terms shall include pluralities and plural terms shall include the singular.

Definitions

As utilized in accordance with the present disclosure, the following terms, unless otherwise indicated, shall be understood to have the following meanings:

The term “isolated polynucleotide” as used herein means a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The term “isolated protein” referred to herein means a protein encoded by genomic DNA, cDNA, recombinant DNA, recombinant RNA, or synthetic origin or some combination thereof, which (1) is free of at least some proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is naturally found when isolated from the source cell, (5) is not linked (by covalent or noncovalent interaction) to all or a portion of a polypeptide to which the “isolated protein” is linked in nature, (6) is operatively linked (by covalent or noncovalent interaction) to a polypeptide with which it is not linked in nature, or (8) does not occur in nature. Preferably, the isolated protein is substantially free from other contaminating proteins or polypeptides or other contaminants that are found in its natural environment that would interfere with its therapeutic, diagnostic, prophylactic or research use.

The terms “polypeptide” or “protein” is used herein to refer to native proteins, that is, proteins produced by naturally-occurring and specifically non-recombinant cells, or genetically-engineered or recombinant cells, and comprise molecules having the amino acid sequence of the native protein, or sequences that have deletions, additions, and/or substitutions of one or more amino acids of the native sequence. The terms “polypeptide” and “protein” specifically encompasses FoxM1B, or species thereof that have deletions, additions, and/or substitutions of one or more amino acids of FoxM1B having at least one functional property of the FoxM1B protein.

The term “naturally-occurring” as used herein refers to an object that can be found in nature, for example, a polypeptide or polynucleotide sequence that is present in an organism (including a virus) that can be isolated from a source in nature and which has not been intentionally modified by man. The term “naturally occurring” or “native” when used in connection with biological materials such as nucleic acid molecules, polypeptides, host cells, and the like, refers to materials which are found in nature and are not manipulated by man. Similarly, “recombinant,” “non-naturally occurring” or “non-native” as used herein refers to a material that is not found in nature or that has been structurally modified or synthesized by man.

As used herein, the twenty conventional amino acids and their abbreviations follow conventional usage. See IMMUNOLOGY—A SYNTHESIS, 2nd Edition, (E. S. Golub and D. R. Gren, Eds.), 1991, Sinauer Associates, Sunderland, Mass., which is incorporated herein by reference for any purpose. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution does not substantially change the structural characteristics of the parent sequence (e.g., a replacement amino acid should not disrupt secondary structure that characterizes the parent or native protein, such as a helix). Examples of art-recognized polypeptide secondary and tertiary structures are described in PROTEINS, STRUCTURES AND MOLECULAR PRINCIPLES (Creighton, Ed.), 1984, W. H. New York: Freeman and Company; INTRODUCTION TO PROTEIN STRUCTURE (Branden and Tooze, eds.), 1991, New York: Garland Publishing; and Thornton et at., 1991, Nature 354: 105, which are each incorporated herein by reference.

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, non-conservative substitutions may involve the exchange of a member of one of these classes for a member from another class. Such substituted residues may be introduced into regions of the human antibody that are homologous with non-human antibodies, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte et al, 1982, J Mol. Biol. 157:105-131).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen-binding or immunogenicity, i.e., with a biological property of the protein.

As described in U.S. Pat. No. 4,554,101, the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Exemplary amino acid substitutions are set forth in Table 1.

TABLE 1
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val,Met,Ala, | Leu |
| | Phe, Norleucine | |
| Leu | Norleucine, Ile, | Ile |
| | Val, Met, Ala, Phe | |
| Lys | Arg, Gln, Asn, | Arg |
| | 1,4 Diamine-butyric Acid | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan can determine suitable variants of the polypeptide as set forth herein using well-known techniques. In certain embodiments, one skilled in the art can identify suitable areas of the molecule that can be changed without destroying activity by targeting regions not believed to be important for activity. In certain embodiments, one can identify residues and portions of the molecules that are conserved among similar polypeptides. In certain embodiments, even areas that are important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants can be used to gather information about suitable variants. For example, if it was discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

Stereoisomers (e.g., D-amino acids) of the twenty conventional amino acids, non-naturally occurring amino acids such as α,α-disubstituted amino acids, N-alkyl amino acids, lactic acid, and other unconventional amino acids may also be suitable components for polypeptides of the present invention. Examples of unconventional amino acids include but are not limited to: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Peptide analogs are commonly used in the pharmaceutical industry as non-peptide drugs with properties analogous to those of the template peptide. These types of non-peptide compound are termed “peptide mimetics” or “peptidomimetics.” (See Fauchere, 1986, Adv. Drug Res. 15: 29; Veber and Freidinger, 1985, TINS p.392; and Evans et al., 1987, J. Med. Chem. 30: 1229, which are incorporated herein by reference for any purpose.) Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage such as: —CH₂NH—, —CH₂S—, —CH₂—CH₂—, —CH═CH— (cis and trans), —COCH₂—,—CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, conformationally-constrained peptides comprising a consensus sequence or a substantially identical consensus sequence variation may be generated by methods known in the art (Rizo and Gierasch, 1992, Ann. Rev. Biochem. 61: 387), incorporated herein by reference for any purpose); for example, by adding internal cysteine residues capable of forming intramolecular disulfide bridges which cyclize the peptide.

Unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

The term “polynucleotide” as used herein means a polymeric form of nucleotides that are at least 10 bases in length. In certain embodiments, the bases may be ribonucleotides or deoxyribonucleotides or a modified form of either type of nucleotide. The term includes single and double stranded forms of DNA.

The term “oligonucleotide” as used herein includes naturally occurring, and modified nucleotides linked together by naturally occurring, and/or non-naturally occurring oligonucleotide linkages. Oligonucleotides are a polynucleotide subset generally comprising no more than 200 nucleotides. In certain embodiments, oligonucleotides are 10 to 60 nucleotides in length. In certain embodiments, oligonucleotides are 12, 13, 14, 15, 16, 17, 18, 19, or 20 to 40 bases in length. Oligonucleotides are single stranded, e.g. for use in the construction of a gene mutant using site directed mutagenesis techniques. Oligonucleotides of the invention may be sense or antisense oligonucleotides.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotides linkages such as phosphate, phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res. 14: 9081; Stec et al., 1984, J. Am. Chem. Soc. 106: 6077; Stein et al, 1988, Nucl. Acids Res. 16: 3209; Zon et al., 1991, Anti-Cancer Drug Design 6: 539; Zon et al., 1991, OLIGONUCLEOTIDES AND ANALOGUES: A PRACTICAL APPROACH, (F. Eckstein, ed.), Oxford University Press, Oxford England, pp. 87-108; Stec et al., U.S. Pat. No. 5,151,510; Uhlmann and Peyman, 1990, Chemical Reviews 90: 543, the disclosures of each of which are hereby incorporated by reference for any purpose. An oligonucleotide can include a detectable label, such as a radiolabel, a fluorescent label, an antigenic label or a hapten.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins can be used that are known in the art. Examples of labels for polypeptides include, but are not limited to, the following: radioisotopes or radionuclides (e.g., 3H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent groups, biotin, and predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths (such as—(CH₂)ₙ—, n=1-50, more preferably 1-20) to reduce steric hindrance.

The phrase “recombinant nucleic acid construct” as used herein refers to a DNA or RNA sequence that comprises a coding sequence that is operatively linked to a control sequence. A recombinant nucleic acid construct of the invention is capable of expressing a protein that is encoded by the coding sequence when introduced into a cell. A recombinant nucleic acid construct of the invention preferably comprises the nucleic acid sequence that encodes a protein as set forth in SEQ ID NO: 2, such as the nucleic acid sequence as set forth in SEQ ID NO: 1, whereby a cell contacted with the recombinant nucleic acid construct expresses FoxM1B protein. The term “operatively linked” as used herein refers to components that are in a relationship permitting them to function in their intended or conventional manner. For example, a control sequence “operatively linked” to a coding sequence is ligated thereto in such a way that expression of the coding sequence is achieved under conditions compatible with the control sequences.

The term “control sequence” as used herein refers to polynucleotide sequences that can effect the expression and processing of coding sequences to which they are ligated. The nature of such control sequences may differ depending upon the host organism. According to certain embodiments, control sequences for prokaryotes may include promoters, repressors, operators, ribosomal binding sites, and transcription termination sequences and antisense mRNA. According to certain embodiments, control sequences for eukaryotes may include promoters, enhancers and transcription termination sequence, protein degradation, mRNA degradation, nuclear localization, nuclear export, cytoplasmic retention, protein phosphorylation, protein acetylation, protein sumolation, RNAi inhibition. In certain embodiments, “control sequences” can include leader sequences and/or fusion partner sequences. “Control sequences” are “operatively linked” to a coding sequence when the “control sequence” effects expression and processing of coding sequences to which they are ligated.

As used herein, the phrase “liver specific promoters” refers to nucleic acid sequences that are capable of directing transcription of a coding sequence and are activated specifically within a liver cell. Liver specific promoters suitable for the methods of the invention include, but are not limited to, human or mouse α1-antitrypsin, albumin promoter, serum amyloid A, transthyretin, and hepatocyte nuclear factor 6.

The term “vector” is used to refer to any molecule (e.g., nucleic acid, plasmid, or virus) used to transfer coding information to a host cell. Viral vectors suitable for the methods of the invention include those derived from, for example, an adenovirus, an adeno-associated virus, a retrovirus, a herpes simplex virus, or a vaccinia virus.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “host cell” is used to refer to a cell into which has been introduced, or that is capable of having introduced, a nucleic acid sequence and then of expressing a gene of interest. The term includes the progeny of the parent cell, whether or not the progeny is identical in morphology or in genetic make-up to the original parent, so long as the gene is present.

The term “transduction” is used to refer to the transfer of genes from one bacterium to another, usually by a phage. “Transduction” also refers to the acquisition and transfer of eukaryotic cellular sequences by viruses such as retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52: 456; Sambrook et al., 2001, ibid.; Davis et al., 1986, BASIC METHODS IN MOLECULAR BIOLOGY (Elsevier); and Chu et al., 1981, Gene 13: 197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is stably transformed when the DNA is replicated with the division of the cell.

The term “pharmaceutical composition” as used herein refers to a chemical compound or composition capable of inducing a desired therapeutic effect when properly administered to a patient.

The term “therapeutically effective amount” refers to the amount of growth hormone or a compound identified in a screening method of the invention determined to produce a therapeutic response in a mammal. Such therapeutically effective amounts are readily ascertained by one of ordinary skill in the art.

As used herein, “substantially pure” means an object species that is the predominant species present (i.e., on a molar basis it is more abundant than any other individual species in the composition). In certain embodiments, a substantially purified fraction is a composition wherein the object species comprises at least about 50 percent (on a molar basis or on a weight or number basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (wherein contaminating species cannot be detected in the composition by conventional detection methods) wherein the composition consists essentially of a single macromolecular species.

The term “patient” includes human and animal subjects.

As used herein, the term “autograft” refers to removal of part of an organism and its replacement in the body of the same individual. An autograft can be the introduction of autologous organs, tissue, or cells in an individual.

As used herein the term “allograft” refers to the removal of part of one individual and its replacement in the body of a different individual. An allograft is also referred to as a xenograft, heterograft, or heterologous graft. Allografts can be obtained, for example, from organ donation.

The phrase “liver cells” as used herein refers to the cells that make up a mammalian liver. Liver cells include, for example, hepatocytes, Kupffer cells, biliary epithelial cells, fenestrated endothelial cells, and cells of Ito.

As used herein, the term “liver regeneration” refers to the growth or proliferation of new liver tissue. Regenerated liver tissue of the invention will have cytological, histological, and functional characteristics of normal liver tissue. Such characteristics can be examined by any method known in the art. For example, regenerated liver tissue of the invention can be examined for expression of common markers indicative of liver function.

The phrase “liver function” refers to one or more of the many physiological functions performed by the liver. Such functions include, but are not limited to, regulating blood sugar levels, endocrine regulation, enzyme systems, interconversion of metabolites (e.g., ketone bodies, sterols and steroids and amino acids); manufacturing blood proteins such as fibrinogen, serum albumin, and cholinesterase, erythropoietic function, detoxification, bile formation, and vitamin storage. Several tests to examine liver function are known in the art, including, for example, measuring alanine amino transferase (ALT), alkaline phosphatase, bilirubin, prothrombin, and albumin.

The phrase “liver disease” or “liver damage” as used herein refers to any condition that impairs liver function. “Liver damage” can occur in response to liver injury caused by any of a number of factors, including, for example, viral infections, parasitic infections, genetic predisposition, autoimmune diseases, exposure to radiation, exposure to hepatotoxic compounds, mechanical injuries, and various environmental toxins. Alcohol, acetominophen, a combination of alcohol and acetominophen, inhalation anaesthetics, niacin, and the herbal supplement kava are some examples of compounds that can cause liver damage. Most forms of liver damage lead to cirrhosis. Cirrhosis is a pathological condition associated with chronic liver damage that includes extensive fibrosis and regenerative nodules. “Fibrosis” as used herein refers to the proliferation of fibroblasts and the formation of scar tissue in the liver.

Common “liver diseases” include, but are not limited to, Reye's syndrome in young children, Wilson's disease, hemochromatosis, alpha-1-antitrypsin deficiency, various parasitic infections, viral diseases, cirrhosis, and liver cancer. Examples of viral diseases include infection by hepatitis A, hepatitis B, hepatitis C, hepatitis D, hepatitis E, and hepatitis G. Examples of parasitic infections include Schistosoma mansoni, Schistosoma hematobium, and Schistosoma japonicum.

The term “growth hormone” refers to growth hormone from any species, including bovine, ovine, porcine, equine, and preferably human, in native-sequence or in variant form, and from any source, whether natural, synthetic, or recombinant. Preferred herein for human use is human native-sequence, mature growth hormone with or without a methionine at its N-terminus. Also preferred is recombinant human growth hormone (hGH), produced, for example, by means of recombinant DNA technology.

Human growth hormone is commercially available and known as somatrem and somatropin. Somatrem is typically used to treat children with growth failure caused by hGH deficiency. The usual weekly dosage of somatrem for children is 0.3 milligram (mg) per kilogram (kg) of body weight. Somatropin is used to treat growth failure caused by Turner's syndrome, kidney disease, or a lack of hGH. The usual weekly dosage of somatropin for children is 0.16 to 0.375 mg per kg of body weight. For adults, 0.006 mg per kg is usually taken daily and increased gradually as needed. AIDS patients experiencing dramatic weight loss are given up to 6 mg of somatropin per day depending on body weight. Somatropin and somatrem are typically administered by injection under the skin or directly into a muscle. Forms of orally administered growth hormone are also known in the art (see, for example, U.S. Pat. No. 6,239,105).

Mouse genetic studies have demonstrated that increased p53 activity results in premature aging and early aging-associated phenotypes (Tyner et al., 2002, Nature 415: 45-53). The potential for increased FoxM1B expression to mediate diminished p53 protein levels in regenerating hepatocytes of old-aged TTR-FoxM1B TG mice was examined as described herein. Prior to hepatocyte DNA replication (24 to 36 hours post PHx), Western blot analysis revealed a 50-70% reduction in p53 protein levels in regenerating livers of old-aged TTR-FoxM1B TG mice compared to old-aged WT mice. Coincident with the reduction of p53 protein levels, a 50% reduction in p21 Cip1 protein expression prior to S-phase in regenerating livers of old-aged TTR-FoxM1B TG mice was observed. These liver regeneration studies indicate that maintaining FoxM1B levels caused diminished expression of p53 and p21 Cip1 proteins during the G1 to S-phase transition in old-aged TTR FoxM1B TG mice, which is consistent with preventing reduced proliferating associated with an aging phenotype.

Proliferation defects during aging leads to diminished muscle mass and thinning of the skin, which is associated with a progressive decline in growth hormone (GH) secretion and serum GH binding protein. GH treated old aged mice exhibited increase in regenerating hepatocyte DNA replication and mitosis to levels found in young regenerating liver. Furthermore, as demonstrated herein, increased expression and nuclear localization of FoxM1B is the mechanism by which GH restores hepatocyte proliferation in regenerating liver of old aged mice. This suggests that GH mediates increased hepatocyte proliferation by restoring FoxM1B expression in regenerating livers of old aged mice.

As discussed herein, short term GH administration can be used to stimulate FoxM1B expression and liver cell proliferation in diseased liver that exhibit defects in liver regeneration. Also, short term GH administration can be effective in live donor transplants of liver to recipient. These are donors that give recipient one of their liver lobes and require regeneration of liver in both donor and recipient. GH administration several days prior to donor and recipient with liver disease can stimulate liver regeneration in the liver of the live donor and in the recipient and allow better prognosis for both patients. The Examples herein demonstrate that GH administration is a useful therapeutic intervention that will enhance liver regeneration through increased expression and nuclear localization of FoxM1B.

The invention provides methods for treating patients diagnosed with liver damage or disease. In these aspects of the invention, patients are treated with growth hormone in a medically acute manner rather than a medically chronic manner, that is, the treatment has a duration that is limited by the nature and extent of the disease, injury or damage and terminates upon detection of positive response in the patient. Preferably, the invention provides transient nuclear localization of FoxM1B protein in the patients treated with growth hormone in a medically acute manner. As used herein, “transient nuclear localization” refers to non-permanent localization of FoxM1B protein in the nucleus of a cell. For example, FoxM1B protein can be induced to localize in the nucleus of a hepatocyte by exposure to growth hormone, while the FoxM1B protein is not detectable in the nucleus once exposure to growth hormone is discontinued.

Patients are preferably screened for liver damage or disease using various assays known in the art. For example, serum levels of liver aminotransferases enzymes (such as alanine aminotransferase (ALT) and aspartate aminotransferase (AST)) can provide an indication of the amount of liver damage in a patient. In most liver diseases, AST levels increase less than ALT (i.e., the ratio of AST/ALT is less than 1). In liver injury caused by alcohol, however, the ratio is often >2. Other tests for determining the amount of liver damage in a patient involve measuring levels of bilirubin, prothrombin, and albumin. For a review of various methods for screening and diagnosing liver damage and disease, see THE MERCK MANUAL, 17^{TH} Edition, (Beers and Berkow, Ed.), 1999, Whitehouse Station, N.J. Thus, patients with, for example, high serum levels of ALT, AST, and bilirubin and with low serum albumin levels advantageously would be administered growth hormone according to the methods of the invention.

For human growth hormone (hGH), a suitable dosage for human administration ranges from 0.001 mg to about 0.2 mg per kg of body weight per day. Generally, therapeutically effective daily dosages of hGH will be from about 0.05 mg to about 0.2 mg per kg of bodyweight per day. For most patients, doses of from 0.07 to 0.15 mg/kg, in one or more applications per day, is effective to obtain the desired result. In an alternative approach, hGH may be administered less frequently, particularly where formulated in a timed-release form, e.g., every other day or every third day for certain indication.

During treatment with hGH, patients can be monitored by the assays described herein and known in the art for improvement in liver function. When liver function is restored to a level that resembles that of a healthy liver, suggesting that liver regeneration process is sufficient, growth hormone administration is discontinued. Thus, it is an advantage of the invention that patients are not chronically exposed to growth hormone.

The methods of the invention are advantageously used with patients having, for example, traumatic liver damage, as well as those who are at high risk for obtaining liver damage, such as alcoholics and those with genetic disposition for liver disease, and those who are regularly exposed to environmental, commercial, and chemical toxins.

In certain embodiments, the invention provides methods for treating liver damage or liver disease in mammals by inducing FoxM1B protein to translocate from the cytoplasm to the nucleus in liver cells, where it potentiates transcription of many cell cycle promotion genes and stimulates cellular proliferation. In a particular embodiment, the mammal is treated with growth hormone to induce nuclear localization of FoxM1B protein.

In other embodiments, the invention provides methods of screening for compounds that induce expression of FoxM1B protein, induce nuclear localization of FoxM1B protein, or induce both expression and nuclear localization of FoxM1B protein. Compounds identified in these screens can be used in the methods of treating liver damage and liver disease as discussed herein.

Screening for compounds the induce expression of FoxM1B protein can be accomplished, for example, with cells that comprise the FoxM1B gene but do not express FoxM1B protein under normal culture conditions. Such cells can include, for example, hepatocytes from aged individuals, host cells comprising the FoxM1B gene as discussed below, or quiescent cells that do not express FoxM1B protein.

The method of screening for compounds that induce expression of FoxM1B in mammalian cells can be accomplished as follows: (a) contacting a plurality of cells that comprise the FoxM1B gene, wherein the FoxM1B protein is not expressed under normal culture conditions, with a candidate compound in the presence of human growth hormone; (b) contacting a plurality of cells that comprise the FoxM1B gene, wherein the FoxM1B protein is not expressed under normal culture conditions, with the candidate compound in the absence of human growth hormone; and (c) assaying FoxM1B expression and localization in the cells from step (a) and step (b); wherein a candidate compound is selected if FoxM1B is localized in the nuclei of cells from step (a) and in the cytoplasm of cells from step (b). Said assay can be a direct assay for nuclear localization of FoxM1B, or can be an indirect assay for the presence or activity of a gene product expressed as a consequence of FoxM1B translocation into the nucleus from the cytoplasm.

The method of screening for compounds that induce nuclear localization of FoxM1B protein can be accomplished by contacting a cell with a candidate compound, wherein the cell expresses FoxM1B protein, and examining localization of FoxM1B protein in the cell. The candidate compound is selected if FoxM1B protein is localized in the nucleus of the cell. In certain embodiments, the Fox M1B is endogenous, i.e., it comprises the genomic DNA complement of the cell. In other embodiments, the FoxM1B is exogenous and is experimentally introduced, most preferably as a recombinant nucleic acid construct of the invention encoding most preferably a heterologous Fox M1B gene, i.e., from a mammalian species different from the host cell species.

The method of screening for compounds that induce both expression and nuclear localization of FoxM1B protein in a manner similar to that of growth hormone, can be accomplished as follows: (a) contacting a plurality of cells that comprise the FoxM1B gene, wherein the FoxM1B protein is not expressed under normal culture conditions, with a candidate compound; y and (b) assaying FoxM1B expression and localization in the cells from step (a); wherein a candidate compound is selected if FoxM1B is expressed and localized in the nuclei of cells contacted with the compound in a manner similar to the pattern observed in cells contacted with growth hormone. In alternative embodiments, the cells of step (a) can be contacted with growth hormone prior to assay in step (b).

Assaying for nuclear localization and expression of FoxM1B protein can be accomplished by any method known the art. For example, immunohistochemistry using anti-FoxM1B antibodies and secondary antibodies labeled with fluorescent markers, such as fluorescein isothiocyanate (FITC), can be used to visualize FoxM1B protein localization by fluorescence microscopy. Alternatively, the primary antibody can be labeled, with a fluorescent label or otherwise. Alternative labels, such as radioactive, enzymatic and hapten labels, are within the scope of this invention.

In certain embodiments, methods of the invention comprise expressing FoxM1B protein in a host cell by introducing into the cell a recombinant nucleic acid construct of the invention. According to such embodiments, the cells are transformed with the recombinant nucleic acid construct using any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference for any purpose). In certain embodiments, the transformation procedure used may depend upon the host to be transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant nucleic acid constructs of the invention typically comprise a nucleic acid molecule encoding the amino acid sequence of FoxM1B protein that is inserted into an appropriate expression vector using standard ligation techniques. Preferably, the recombinant nucleic acid construct of the invention comprises the nucleic acid sequence that encodes a protein as set forth in SEQ ID NO: 2. The vector is typically selected to be functional in the particular host cell employed (i.e., the vector is compatible with the host cell machinery, permitting amplification and/or expression of the gene can occur). For a review of expression vectors, see Nolan and Shatzman, 1998, Curr. Opin. Biotechnol. 9:447-450.

Typically, expression vectors used in any of the host cells contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using in vitro amplification methods such as polymerase chain reaction (PCR) and/or by screening a genomic library with a suitable oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose is readily apparent to one of ordinary skill in the art.

Optionally, the vector may contain a “tag”-encoding sequence, i.e., an oligonucleotide molecule located at the 5′ or 3′ end of the FoxM1B polypeptide coding sequence, the oligonucleotide sequence encoding polyHis (such as hexaHis), or another “tag” for which commercially available antibodies exist, such as FLAG, HA (hemaglutinin influenza virus), or myc. This tag is typically fused to the polypeptide upon expression of the polypeptide, and can serve as a means for affinity purification of the FoxM1B polypeptide from the host cell. Affinity purification can be accomplished, for example, by column chromatography using antibodies against the tag as an affinity matrix. Optionally, the tag can subsequently be removed from the purified FoxM1B polypeptide by various means such as using certain peptidases for cleavage.

An origin of replication is typically a part of prokaryotic expression vectors, particularly those that are commercially available, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various origins (e.g., SV40, polyoma, adenovirus, vesicular stomatitus virus (VSV), or papillomaviruses such as HPV or BPV) are useful for cloning vectors in mammalian cells. Generally, a mammalian origin of replication is not needed for mammalian expression vectors (for example, the SV40 origin is often used only because it contains the early promoter).

A transcription termination sequence is typically located 3′ of the end of a polypeptide-coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein. In eukaryotes, the sequence AAUAAA functions both as a transcription termination signal and as a poly A signal required for endonuclease cleavage and followed by the addition of poly A residues (200 A residues). A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Preferred selectable markers are the kanamycin resistance gene, the ampicillin resistance gene, and the tetracycline resistance gene. A bacterial neomycin resistance gene can also be used most advantageously for selection in both prokaryotic and eukaryotic host cells.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, for example where glycosylation is desired in a eukaryotic host cell expression system, various presequences can be manipulated to improve glycosylation or yield. For example, the peptidase cleavage site of a particular signal peptide can be altered, or pro-sequences added, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated yet active form of the desired polypeptide, if the enzyme cuts at such area within the mature polypeptide.

The expression and cloning vectors of the present invention will typically contain a promoter that is recognized by the host organism and operatively linked to nucleic acid encoding the FoxM1B protein. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no experimental control over gene expression. A large number of promoters, recognized by a variety of potential host cells, are well known. A suitable promoter is operatively linked to the DNA encoding FoxM1B protein by removing the promoter from the source DNA by restriction enzyme digestion or amplifying the promoter by polymerase chain reaction and inserting the desired promoter sequence into the vector.

Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Particular promoters useful in the practice of the recombinant expression vectors of the invention include, but are not limited to: the SV40 early promoter region (Bernoist and Chambon, 1981, Nature 290: 304-10); the CMV promoter; the promoter contained in the 3′ long terminal repeat of Rous sarcoma virus (Yamamoto, et al., 1980, Cell 22: 787-97); the herpes thymidine kinase promoter (Wagner et al., 1981, Proc. Natl. Acad. Sci. U.S.A. 78:1444-45); the regulatory sequences of the metallothionine gene (Brinster et al., 1982, Nature 296: 39-42). Also of interest are the following animal transcriptional control regions, which exhibit tissue specificity and have been utilized in transgenic animals: the elastase I gene control region that is active in pancreatic acinar cells (Swift et al., 1984, Cell 38: 639-46; Ornitz et al., 1986, Cold Spring Harbor Symp. Quant. Biol. 50: 399-409; MacDonald, 1987, Hepatology 7: 425-515); the insulin gene control region that is active in pancreatic beta cells (Hanahan, 1985, Nature 315: 115-22); the mouse mammary tumor virus control region that is active in testicular, breast, lymphoid and mast cells (Leder et al., 1986, Cell 45: 485-95); the beta-globin gene control region that is active in myeloid cells (Mogram et al., 1985, Nature 315: 338-40; Kollias et al., 1986, Cell 46: 89-94); the myelin basic protein gene control region that is active in oligodendrocyte cells in the brain (Readhead et al., 1987, Cell 48: 703-12); the myosin light chain-2 gene control region that is active in skeletal muscle (Sani, 1985, Nature 314: 283-86); the gonadotropic releasing hormone gene control region that is active in the hypothalamus (Mason et al, 1986, Science 234: 1372-78); and most particularly the immunoglobulin gene control region that is active in lymphoid cells (Grosschedl et al., 1984, Cell 38: 647-58; Adames et al., 1985, Nature 318: 533-38; Alexander et al., 1987, Mol. Cell Biol. 7: 1436-44).

Preferably, the promoter of a recombinant nucleic acid construct of the invention is active in the liver. For example, the albumin gene control region is active in liver (Pinkert et al., 1987, Genes and Devel. 1: 268-76); the alpha-feto-protein gene control region is active in liver (Krumlauf et al., 1985, Mol. Cell Biol. 5: 1639-48; Hammer et al., 1987, Science 235: 53-58); and the alpha 1-antitrypsin gene control region is active in the liver (Kelsey et al., 1987, Genes and Devel. 1: 161-71).

An enhancer sequence may be inserted into the vector to increase the transcription of a nucleic acid encoding a FoxM1B protein by higher eukaryotes. Enhancers are cis-acting elements of DNA, usually about 10-300 bp in length, that act on promoters to increase transcription. Enhancers are relatively orientation and position independent. They have been found within introns as well as both within several kilobases 5′ and 3′ to the transcription unit. Several enhancer sequences available from mammalian genes are known (e.g., globin, elastase, albumin, alpha-feto-protein, insulin, transthyretin, and HNF-6). An enhancer from a virus can be used if increased expression of gene is desired. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to a nucleic acid molecule, it is typically located at a site 5′ from the promoter.

Expression vectors of the invention may be constructed from a convenient starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding a FoxM1B protein has been inserted into the proper site of the vector, the completed vector may be inserted into a suitable host cell for amplification and/or polypeptide expression. The transformation of an expression vector for a FoxM1B protein into a selected host cell may be accomplished by well-known methods including methods such as transfection, infection, calcium chloride, electrop oration, microinjection, lipofection, DEAE-dextran method, or other known techniques. The method selected will in part be a function of the type of host cell to be used. These methods and other suitable methods are well known to the skilled artisan, and are set forth, for example, in Sambrook et al., ibid.

The host cell, when cultured under appropriate conditions, synthesizes a FoxM1B protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), such as Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels of FoxM1B protein.

In certain embodiments, the invention provides pharmaceutical compositions comprising a therapeutically effective amount of a compound that induces FoxM1B expression, nuclear localization or expression and or nuclear localization in mammalian liver cells together with a pharmaceutically acceptable diluent, carrier, solubilizer, emulsifier, preservative and/or adjuvant. In other embodiments, the invention provides pharmaceutical compositions that comprise a therapeutically effective amount of a compound that induces FoxM1B expression in mammalian liver cells and also induces FoxM1B protein to translocate into the nucleus of mammalian liver cells together with a pharmaceutically acceptable diluent, carrier, solubilizer, emulsifier, preservative and/or adjuvant. Such compounds are preferably identified in screening methods of the invention.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, trimethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. REMINGTON'S PHARMACEUTICAL SCIENCES, 18^{th} Edition, (A. R. Gennaro, ed.), 1990, Mack Publishing Company.

Optimal pharmaceutical compositions can be determined by one skilled in the art depending upon, for example, the intended route of administration, delivery format and desired dosage. See, for example, REMINGTON'S PHARMACEUTICAL SCIENCES, ibid. Such compositions may influence the physical state, stability, rate of in vivo release and rate of in vivo clearance of the antibodies of the invention.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefor. Pharmaceutical compositions of the invention may be prepared for storage by mixing the selected composition having the desired degree of purity with optional formulation agents (REMINGTON'S PHARMACEUTICAL SCIENCES, ibid.) in the form of a lyophilized cake or an aqueous solution. Further, the FoxM1B-inducing product may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical compositions of the invention can be delivered parenterally. When parenteral administration is contemplated, the therapeutic compositions for use in this invention may be in the form of a pyrogen-free, parenterally acceptable aqueous solution comprising FoxM1B protein or the desired compound identified in a screening method of the invention in a pharmaceutically acceptable vehicle. A particularly suitable vehicle for parenteral injection is sterile distilled water in which the compound identified in a screening method of the invention or FoxM1B protein is formulated as a sterile, isotonic solution, properly preserved. Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which may then be delivered via a depot injection. Formulation with hyaluronic acid has the effect of promoting sustained duration in the circulation. Implantable drug delivery devices may be used to introduce the desired molecule.

Administering FoxM1B protein to a patient can be used for short-term stimulation of liver cell proliferation, for example, in a recipient of a liver transplant. In addition, FoxM1B protein can be administered to a liver donor after the liver or a portion thereof is removed to stimulate liver regeneration to reestablish organ function.

The compositions may be selected for inhalation. In these embodiments, a compound identified in a screening method of the invention or FoxM1B protein is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins.

The pharmaceutical compositions of the invention can be delivered through the digestive tract, such as orally. The preparation of such pharmaceutically acceptable compositions is within the skill of the art. FoxM1B protein or compounds of the invention that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the FoxM1B protein or compound identified in a screening method of the invention. Diluents, flavorings, low melting point waxes, vegetable oils, lubricants, suspending agents, tablet disintegrating agents, and binders may also be employed.

A pharmaceutical composition may involve an effective quantity of FoxM1B protein or a compound identified in a screening method of the invention in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are evident to those skilled in the art, including formulations involving FoxM1B protein or compounds of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT Application No. PCT/US93/00829, which describes the controlled release of porous polymeric microparticles for the delivery of pharmaceutical compositions. Sustained-release preparations may include semipermeable polymer matrices in the form of shaped articles, e.g. films, or microcapsules, polyesters, hydrogels, polylactides (U.S. Pat. No. 3,773,919 and EP 058,481), copolymers of L-glutamic acid and gamma ethyl-L-glutamate (Sidman et al., 1983, Biopolymers 22: 547-556), poly (2-hydroxyethyl-methacrylate) (Langer et al., 1981, J. Biomed. Mater. Res. 15: 167-277) and Langer, 1982, Chem. Tech. 12: 98-105), ethylene vinyl acetate (Langer et al., ibid.) or poly-D(−)-3-hydroxybutyric acid (EP 133,988). Sustained release compositions may also include liposomes, which can be prepared by any of several methods known in the art. See e.g., Eppstein et al., 1985, Proc. Natl. Acad. Sci. USA 82: 3688-3692; EP 036,676; EP 088,046 and EP 143,949.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The present invention is directed to kits for producing a single-dose administration unit. Kits according to the invention may each contain both a first container having a dried protein compound identified in a screening method of the invention and a second container having an aqueous formulation, including for example single and multi-chambered pre-filled syringes (e.g., liquid syringes, lyosyringes or needle-free syringes).

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

The dosing frequency will depend upon the pharmacokinetic parameters of the FoxM1B protein or compound identified in a screening method of the invention in the formulation. For example, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intraparenchymal), intracerebroventricular, intramuscular, intra-ocular, intraarterial, intraportal, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In certain embodiments, it may be desirable to use FoxM1B protein, FoxM1B encoding recombinant nucleic acid constructs or pharmaceutical compositions of compounds identified in a screening method of the invention in an ex vivo manner. In such instances, cells, tissues or organs that have been removed from the patient are exposed to pharmaceutical compositions of the invention or a recombinant nucleic acid construct of the invention comprising the FoxM1B gene after which the cells, tissues and/or organs are subsequently implanted back into the patient.

In certain embodiments, FoxM1B protein, FoxM1B encoding recombinant nucleic acid constructs or pharmaceutical compositions of compounds identified in a screening method of the invention can be delivered by implanting certain cells that have been genetically engineered, using methods such as those described herein, to express and secrete the polypeptide. Such cells may be animal or human cells, and may be autologous, heterologous, or xenogeneic, or may be immortalized. In order to decrease the chances of an immunological response, the cells may be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

Pharmaceutical compositions of the invention can be administered alone or in combination with other therapeutic agents, in particular, in combination with other cancer therapy agents. Such agents generally include radiation therapy or chemotherapy. Chemotherapy, for example, can involve treatment with one or more of the following: anthracyclines, taxol, tamoxifene, doxorubicin, 5-fluorouracil, and other drugs known to one skilled in the art.

One approach for increasing, or causing, the expression of FoxM1B polypeptide from a cell's endogenous FoxM1B gene involves increasing, or causing, the expression of a gene or genes (e.g., transcription factors) and/or decreasing the expression of a gene or genes (e.g., transcriptional repressors) in a manner which results in de novo or increased FoxM1B polypeptide production from the cell's endogenous FoxM1B gene. This method includes the introduction of a non-naturally occurring polypeptide (e.g., a polypeptide comprising a site specific DNA binding domain fused to a transcriptional factor domain) into the cell such that de novo or increased FoxM1B polypeptide production from the cell's endogenous FoxM1B gene results.

The present invention further relates to DNA constructs useful in the method of altering expression of a target gene. In certain embodiments, the exemplary DNA constructs comprise: (a) one or more targeting sequences, (b) a regulatory sequence, (c) an exon, and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of FoxM1B polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a FoxM1B polypeptide, which nucleotides may be used as targeting sequences.

FoxM1B polypeptide cell therapy, e.g., the implantation of cells producing FoxM1B polypeptides, is also contemplated. This embodiment involves implanting cells capable of synthesizing and secreting a biologically active form of FoxM1B polypeptide. Such FoxM1B polypeptide-producing cells can be cells that are natural producers of FoxM1B polypeptides or may be recombinant cells whose ability to produce FoxM1B polypeptides has been augmented by transformation with a gene encoding the desired FoxM1B polypeptide or with a gene augmenting the expression of FoxM1B polypeptide. Such a modification may be accomplished by means of a vector suitable for delivering the gene as well as promoting its expression and secretion. In order to minimize a potential immunological reaction in patients being administered an FoxM1B polypeptide, as may occur with the administration of a polypeptide of a foreign species, it is preferred that the natural cells producing FoxM1B polypeptide be of human origin and produce human FoxM1B polypeptide. Likewise, it is preferred that the recombinant cells producing FoxM1B polypeptide be transformed with an expression vector containing a gene encoding a human FoxM1B polypeptide.

Implanted cells may be encapsulated to avoid the infiltration of surrounding tissue. Human or non-human animal cells may be implanted in patients in biocompatible, semipermeable polymeric enclosures or membranes that allow the release of FoxM1B polypeptide, but that prevent the destruction of the cells by the patient's immune system or by other detrimental factors from the surrounding tissue. Alternatively, the patient's own cells, transformed to produce FoxM1B polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (PCT Pub. No. WO 95/05452 and PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for the delivery of the molecules from living cells to specific sites within a recipient. In addition, see U.S. Pat. Nos. 4,892,538; 5,011,472; and 5,106,627. A system for encapsulating living cells is described in PCT Pub. No. WO 91/10425 (Aebischer et al.). See also, PCT Pub. No. WO 91/10470 (Aebischer et al.); Winn et al, 1991, Exper. Neurol. 113:322-29; Aebischer et al., 1991, Exper. Neurol. 111 :269-75; and Tresco et al., 1992, ASAIO 38:17-23.

In vivo, ex vivo and in vitro gene therapy delivery of FoxM1B polypeptides is also provided herein. One example of a gene therapy technique is to use the FoxM1B gene (either genomic DNA, cDNA, and/or synthetic DNA) encoding a FoxM1B polypeptide that can be operatively linked to a constitutive or inducible promoter to form a “gene therapy DNA construct.” The promoter may be homologous or heterologous to the endogenous FoxM1B gene, provided that it is active in the cell or tissue type into which the construct is inserted. Other components of the gene therapy DNA construct may optionally include DNA molecules designed for site-specific integration (e.g., endogenous sequences useful for homologous recombination), tissue-specific promoters, enhancers or silencers, DNA molecules capable of providing a selective advantage over the parent cell, DNA molecules useful as labels to identify transformed cells, negative selection systems, cell specific binding agents (as, for example, for cell targeting), cell-specific internalization factors, transcription factors enhancing expression from a vector, and factors enabling vector production.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the FoxM1B gene in the target cell. Such elements are turned on in response to an appropriate effector. In this way, a therapeutic polypeptide can be expressed when desired. One conventional control means involves the use of small molecule dimerizers or rapalogs to dimerize chimeric proteins which contain a small molecule-binding domain and a domain capable of initiating a biological process, such as a DNA-binding protein or transcriptional activation protein (see PCT Pub. Nos. WO 96/41865, WO 97/31898, and WO 97/31899). The dimerization of the proteins can be used to initiate transcription of the transgene.

In vivo gene therapy may be accomplished by introducing the gene encoding FoxM1B polypeptide into cells via local delivery of a FoxM1B nucleic acid molecule, by direct injection or by other appropriate viral or non-viral delivery vectors. (Hefti, 1994, Neurobiology 25:1418-35.) For example, a nucleic acid molecule encoding a FoxM1B polypeptide may be contained in an adeno-associated virus (AAV) vector for delivery to the targeted cells (see, e.g., Johnson, PCT Pub. No. WO 95/34670; PCT App. No. PCT/US95/07178). The recombinant AAV genome typically contains AAV inverted terminal repeats flanking a DNA sequence encoding a FoxM1B polypeptide operatively linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. Nos. 5,631,236 (involving adenoviral vectors), 5,672,510 (involving retroviral vectors), 5,635,399 (involving retroviral vectors expressing cytokines).

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. Nos. 4,970,154 (involving electroporation techniques), 5,679,559 (describing a lipoprotein-containing system for gene delivery), 5,676,954 (involving liposome carriers), 5,593,875 (describing methods for calcium phosphate transfection), and 4,945,050 (describing a process wherein biologically active particles are propelled at cells at a speed whereby the particles penetrate the surface of the cells and become incorporated into the interior of the cells), and PCT Pub. No. WO 96/40958 (involving nuclear ligands).

It is also contemplated that FoxM1B gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

Another means of increasing endogenous FoxM1B polypeptide expression in a cell via gene therapy is to insert one or more enhancer elements into the FoxM1B polypeptide promoter, where the enhancer elements can serve to increase transcriptional activity of the FoxM1B gene. The enhancer elements used are selected based on the tissue in which one desires to activate the gene—enhancer elements known to confer promoter activation in that tissue are selected. For example, if a gene encoding a FoxM1B polypeptide is to be “turned on” in T-cells, the lck promoter enhancer element may be used. Here, the functional portion of the transcriptional element to be added may be inserted into a fragment of DNA containing the FoxM1B polypeptide promoter (and optionally, inserted into a vector and/or 5′ and/or 3′ flanking sequences) using standard cloning techniques. This construct, known as a “homologous recombination construct,” can then be introduced into the desired cells either ex vivo or in vivo.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### Example 1

Effects of Increased FoxM1B Expression on DNA Replication and Mitosis in Regenerating Liver of Aged Transgenic Mice

Transgenic CD-1 mice were generated using the −3 kb transthyretin (TTR) promoter to constitutively express the FoxM1B transgene (SEQ ID NO: 1 as shown in FIG. 1) in hepatocytes as described (Ye et al., 1999, Mol. Cell Biol., 19: 8570-8580). Twelve-month old wild type CD-1 (WT) and TTR-FoxM1B (TG) mice were anesthetized with methoxyflurane (Metofane; Schering-Plough Animal Health Corp., Union, N.J.) and the left lateral, left median, and right median lobes of the liver were removed following midventral laparotomy to induce liver regeneration (Higgins et al., 1931, Arch. Pathol. 12:186-202). Removal of the gallbladder, located between the left and right median lobes was carefully avoided. Following surgery, animals were given one subcutaneous injection of ampicillin (50 μg/g body weight) in saline. Two hours prior to harvesting the remnant liver, animals were injected intraperitoneally with 10 mg/mL of 5-bromo-2′-deoxyuridine (BrdU; 50 μg/g body weight) in phosphate-buffered saline (PBS). Two mice were sacrificed by CO₂ asphyxiation at 24, 32, 36, 40, 44, and 48 hours after partial hepatectomy (PHx) surgery and their livers were removed. The dissected livers were divided into three portions: one for paraffin embedding, one for total RNA isolation, and one for total protein isolation.

Liver portions for paraffin embedding were fixed in 4% paraformaldehyde overnight and embedded in paraffin. Tissues were cut into 5 μm sections with a microtome and fixed onto slides. Sections were dewaxed with xylenes, rehydrated with decreasing graded ethanol washes, and placed in PBS with 0.25% Triton X-100 (PBT). A microwave antigen-retrieval method was used to enhance antigenic reactivity of the antibodies as previously described (Zhou et al., 1996, J. Histochem. Cytochem. 44:1183-1193). Sections were immunohistochemically stained with anti-BrdU monoclonal antibodies according to the manufacturer's instructions (Boehringer Mannheim). The number of BrdU positive nuclei per 1000 hepatocytes was counted and the mean BrdU positive cells and standard deviation (SD) were calculated using two regenerating liver samples from each time point. Regenerating livers from 2 month old (young) CD-1 mice were examined and included as a comparison. The 2 month old livers display an S-phase peak at 40 hours after PHx (FIG. 2). A much smaller 40-hour S-phase peak was observed in the regenerating livers from 12 month old WT mice (FIG. 2). The regenerating livers of 12 month old TG mice exhibited a sharp S-phase peak at 40 hours similar to that observed in the 2 month old livers (FIG. 2). Immunohistochemical staining with anti-BrdU antibodies shows the increase in BrdU incorporation in the TG livers compared with the WT livers at 40 hours. In addition, at 48 hours post PHx, the regenerating hepatocytes of the old WT mice displayed fewer mitotic figures compared with those of the TG mice (FIG. 3).

These studies demonstrate that increased hepatocyte expression of FoxM1B in regenerating livers of old-aged transgenic mice stimulated hepatocyte DNA replication and mitosis to levels found in young regenerating mouse liver.

### Example 2

The Effects of PHx on the Levels of FoxM1B mRNA and Protein Expression in Young and Old WT Mice and Old TG Mice.

Total RNA from regenerating livers of wild type (WT) and transgenic (TG) mice was extracted 24, 32, 36, 40, and 44 hours post partial hepatectomy (PHx) by an acid guanidium thiocyanate-phenol-chloroform extraction method with RNA-STAT-60 (Tel-Test “B” Inc., Friendswood, Tex.). Antisense RNase protection probes for the human and mouse FoxM1B transgene and for mouse cyclophilin were generated as described (Ye et al., 1997, Mol. Cell Biol. 17:1626-1641; Wang et al., 2001, Hepatology 33:1404-1414). RNase protection assays were performed by hybridizing 20 to 40 μg of total liver RNA with {³²P} UTP-labeled probes followed by digestion with RNase One, electrophoresis, and autoradiography as described previously (Ye et al., 1997, Mol. Cell Biol. 17:1626-1641; Wang et al., 2001, Hepatology 33:1404-1414; Rausa et al., 2000, Mol. Cell Biol. 20:8264-8282). The X-ray films were scanned and the BioMax 1D program (Eastman Kodak Co) was used to quantify expression levels, which were normalized to cyclophilin RNA levels. FoxM1B mRNA levels were induced at 40 hours, consistent with the S-phase peak, in the regenerating liver from 2 month old WT mice (FIG. 4A, FIG. 2). Likewise, the S-phase peak observed in old TG mice at 40 hours post PHx was accompanied by elevated FoxM1B mRNA (FIG. 4B). Induction of FoxM1B mRNA at 40 hours was diminished in 12 month old WT mice compared with the young mice (FIGS. 4A and B).

Total protein extracts from regenerating livers of 12 month old TG and WT mice at 24, 32, 36, 40, and 44 hours after PHx were isolated as described (Rausa et al., 2000, Mol. Cell Biol. 20: 8264-8282). Western blot analysis was done by separating 50 μg of total liver protein by SDS-PAGE, transferring to Protran membrane (Schleicher & Schuell, Keene, NH), incubating with HFH-11 (FoxM1B) antibody (Ye et al., 1997, Mol. Cell Biol. 17: 1626-1641; Ye et al., 1999, Mol. Cell Biol. 19: 8570-8580), and amplifying the signal with biotin conjugated anti-rabbit IgG (BioRad, Hercules, Calif.). Signal was detected with enhanced chemiluminesence (ECL, Amersham Pharmacia Biotech, Piscataway, N.J.). Elevated protein levels of FoxM1B were associated with increased BrdU incorporation and FoxM1B mRNA expression at 40 hours after PHx (FIGS. 3, 4C, and 5). No increase in FoxM1B protein expression was observed in regenerating hepatocytes of old-aged WT mice (FIG. 5).

These studies demonstrate that increased FoxM1B mRNA and protein levels in transgenic mice is associated with increased hepatocyte proliferation in regenerating liver of old-aged transgenic mice. Example 3

Altered Expression of Genes Involved in S-Phase and M-Phase Progression in Response to Increased Expression of FoxM1B in Regenerating Livers

RNase protection probes for Cyclin D1, Cyclin D3, Cyclin E, Cyclin Al, Cyclin A2, Cyclin B1, Cyclin B2, and Cyclin F were purchased from Pharmingen (San Diego, Calif.) and probes for Cdc25B and p55Cdc were purchased from Clontech. RNase protection assays were performed for Cyclin genes using procedures described by the manufacturer and for other genes as described above on 20-40 μg of total liver RNA isolated from WT and TG mice 24, 32, 36, 40, and 44 hours after PHx. The expression of Cyclin Dl gene, which promotes S-phase, was elevated in the aged TG mice at 36 to 40 hours post PHx, just before the initiation of hepatocyte DNA replication (FIG. 6). Expression levels of Cyclin E were also increased at 40 hours post PHx in old TG mice (FIG. 6). The induction of Cyclin D1 and Cyclin E in the regenerating livers of TG mice is associated with increased expression of FoxM1B. Cyclin D1 and Cyclin E expression was decreased during the G1/S transition of the cell cycle of regenerating livers of old WT mice (FIG. 6). In addition, elevated FoxM1B levels led to increased expression of Cyclin A2 in these livers (FIG. 6). The data show that restoring FoxM1B expression in regenerating liver of old mice stimulates the induction of Cyclin D1, Cyclin E, and Cyclin A2, which facilitate hepatocyte entry and progression through S-phase.

During the peak of hepatocyte DNA replication, a significant induction of Cyclin B1 and Cyclin B2 was observed only in the regenerating liver from old TG mice (FIG. 6). Also at this time point, Cyclin F levels were increased significantly in the regenerating liver of 12 month old TG mice (FIG. 6). Greater activation of Cdc25B mRNA was observed between 40 and 44 hours post PHx in the liver of TG animals than in the liver of WT animals (FIG. 6). In addition, only the liver of TG animals displayed induced expression of p55Cdc after PHx (FIG. 6). Cyclin B1 and Cyclin B2 mediate cell cycle progression from the G2 phase into mitosis (Zachariae et al., 1999, Genes Dev. 13: 2039-2058). Cyclin F is essential for M-phase progression because it facilitates nuclear translocation of the Cyclin B complexes (Kong et al., 2000, EMBO J 19: 1378-1388). M-phase progression is also mediated by Cdc25B, which activates the mitotic kinase cdk1/cyclin B (Sebastian et al., 1993, Proc. Natl. Acad. Sci. USA 90: 3521-3524; Trembley et al., 1996, Cell Growth Differ. 7: 903-916; Nilsson et al., 2000, Prog. Cell Cycle Res. 4: 107-114). Degradation of Cyclin proteins, a process necessary for completion of mitosis, is regulated by p55Cdc (Zachariae et al., 1999, Genes Dev. 13: 2039-2058).

These results demonstrate that increased expression of FoxM1B in old TG mice induces M-phase promoting genes including Cyclin B1, Cyclin B2, Cyclin F, Cdc25B, and p55Cdc.

### Example 4

p21 and p53 Expression in the Liver of Old FoxM1B Transgenic Mice After Partial Hepatectomy

Twenty to forty micrograms of total liver RNA was isolated from old TG and WT mice 24, 32, 36, and 40 hours after PHx. An RNase protection probe for p21 was received as a gift from Dr. Guy Adami (University of Illinois at Chicago). As above, approximately 2×10⁵ cpm of each probe was hybridized at 45 C or 55 C to 20 μg of total RNA in a solution containing 20 mM PIPES (pH 6.4), 400 mM NaCl, 1 mM EDTA and 80% formamide overnight. After hybridization, samples were digested for 1 hr at 37 C by using 10 units per sample of RNase One enzyme according to the manufacturer's protocol (Promega, Madison, Wis.). The RNase One protected fragments were electrophoresed on an 8% polyacrylamide-8M urea gel, followed by autoradiography. Quantitation of expression levels was determined with scanned X-ray films by using the BioMax ID program (Eastman Kodak, Rochester, N.Y.). The cyclophilin hybridization signal was used for normalization control between different liver RNA samples. p21 mRNA levels were decreased during the G1/S transition of the cell cycle in the old TG animals (FIG. 7, 32 to 40 hours post PHx).

Paraffin embedded tissue samples from regenerating livers of 12 month old WT and TG mice dissected 24, 32, and 40 hours post PHx were sectioned with a microtome and prepared for immunohistochemical staining as described above. Sections were incubated with anti-p21 antibodies (Oncogene Science, Cambridge, Mass.) or anti-FoxM1B antibodies and detected using the ABC kit and DAB peroxidase substrate according to manufacturer's instructions (Vector Laboratories, Burlingame, Calif.). The number of p21 positive and FoxM1B positive hepatocytes per 1000 nuclei for each mouse liver was determined, and data from two mice for each time point were used to calculate the mean ±standard deviation (SD) using the Analysis ToolPak in Macintosh Microsoft Excel 98. p21 protein levels in the nuclei of regenerating liver of old TG mice were reduced compared with levels observed in the WT liver at 32 hours after PHx (FIG. 8). However, at 36 hours after PHx, p21 nuclear protein levels in liver of TG mice were similar to those in WT liver (FIG. 8), which is consistent with the role of p21 in assembling the Cyclin D/cdk4/6 complex necessary for progression into S-phase (Cheng, et al., 1999, Embo J. 18:1571-1583).

The ability of increased FoxM1B expression to mediate diminished p53 protein levels in regenerating hepatocytes of old-aged TTR-FoxM1B TG mice was also examined. Prior to hepatocyte DNA replication (24 to 36 hours post PHx), Western blot analysis revealed a 50-70% reduction in p53 protein levels in regenerating livers of old-aged TTR-FoxM1B TG mice compared to old-aged WT mice (FIGS. 9A-C). Coincident with the reduction of p53 protein levels, a 50% reduction in p21 Cip1 protein expression prior to S-phase in regenerating livers of old-aged TTR-FoxM1B TG mice was observed.

These liver regeneration studies indicate that maintaining FoxM1B levels caused diminished expression of p53 and p21 Cip1 proteins during the G1 to S-phase transition in old-aged TTR FoxM1B TG mice, which is consistent with preventing reduced proliferating associated with an aging phenotype.

### Example 5

The Effects of Carbon Tetrachloride Induced Liver Injury on Localization of FoxM1B and Hepatocyte DNA Replication in FoxM1B Transgenic Mice

Wild type or FoxM1B transgenic male CD-1 mice (8-10 weeks of age) were given a single intraperitoneal (IP) injection of a 10% solution of carbon tetrachloride (10 μL CCl₄/g body weight; Sigma-Aldrich, St. Louis, Mo.) dissolved in light mineral oil, as described in Serfas et al., 1997, Cell Growth Differ. 8:951-961. Mice were subjected to an IP injection of 10 mg/mL solution of 5-bromo-2′-deoxyuridine (BrdU; 50 μg/g body weight) in phosphate buffered saline (PBS) two hours prior to harvesting the liver as described previously (Ye et al., 1999, Mol. Cell Biol. 19: 8570-8580). Mice were sacrificed by CO₂ asphyxiation at 16, 20, 24, 28, 32, 34, 36, 40, 44, and 48 hour intervals following CCl₄ administration. A portion of liver tissue was used to prepare total RNA and the rest of the liver was paraffin embedded as described previously (Id.). To determine the statistical significance of any observed differences between transgenic and wild type mice four mice were sacrificed at each time point.

Nuclear localization of FoxM1B protein requires proliferative signaling (Id). Therefore, an affinity purified FoxM1B antibody was used as above for immunohistochemical staining of mouse liver sections at the various time points following CCl₄ liver injury. Regenerating WT hepatocytes displayed FoxM1B nuclear staining between 32 to 36 hours following CCl₄ liver injury (FIGS. 10A-B) and reached maximum staining by the 40-hour time point (FIG. 10C). In contrast, nuclear FoxM1B protein staining was found in regenerating TG hepatocytes at the earliest time point examined (20 hours after CCl₄ injury) and persisted throughout the liver regeneration process (FIGS. 10D-F).

The timing of hepatocyte entry into S-phase, DNA synthesis in CCl₄ regenerating liver was examined by immunohistochemical staining of BrdU incorporation into DNA as described above. In WT livers, a few BrdU positive staining hepatocytes were detected at 36 hours after CCl₄ injury, while hepatocyte DNA replication reached a maximum by 40 hours and displayed a broad persistent S-phase peak (FIG. 11). In contrast, TG hepatocytes showed detectable BrdU incorporation at 32 hours after CCl₄ injury, while hepatocyte replication was significantly increased by 34 hours and became maximal by 36 hours (FIG. 11).

These studies show earlier nuclear expression of the FoxM1B transgene protein results in a six-hour acceleration in the onset of hepatocyte DNA replication following liver injury induced by CCl₄.

### Example 6

The Effects of Carbon Tetrachloride Induced Liver Injury on p21 Levels in FoxM1B Transgenic Mice

To determine whether earlier transgenic hepatocyte replication correlates with diminished p21 protein expression, livers of WT and TG mice were removed 16, 20, 24, 28, 32, 36, and 40 hours after CCl₄ induced liver injury and examined by immunohistochemical staining as described above with anti-p21 antibodies. The number of p21 staining periportal hepatocytes present in regenerating TG hepatocytes was significantly decreased between 16 and 36 hours post CCl₄ liver injury compared with regenerating WT hepatocytes (FIG. 12A). The difference in hepatocyte expression of p21 protein was greatest at 36 hours following CCl₄ administration (FIG. 12A), corresponding to the time of maximum TG hepatocyte DNA replication and barely detectable WT hepatocyte replication (FIG. 11). The p21 expression pattern was the same at 40 hours post CCl₄ liver injury when both WT and TG hepatocytes show abundant BrdU incorporation.

The level of p21 mRNA expression was also examined in CCl₄ regenerating livers of TG mice and WT mice. RNase protection assays were performed as described in duplicate. Hepatic p21 mRNA was normalized and is presented graphically, demonstrating that regenerating WT hepatic expression of p21 remained constant throughout the time points considered (FIG. 12B). A significant reduction in TG hepatic levels of p21 mRNA was observed between 28 and 32 hours following CCl₄ liver injury (FIG. 12B), which is consistent with early hepatocyte entry into S-phase as seen in FIG. 11.

These studies demonstrate that diminished expression of p21, which is inhibitory to DNA replication, mediates accelerated hepatocyte proliferation during liver regeneration.

### Example 7

Differential Expression of Proliferation-Specific Genes in Regenerating Livers of Transgenic and Wild Type Mice Following CCl₄ Liver Injury

As described above, RNase protection assays were performed with Cyclin genes using RNA protection probes and a kit made by Pharmingen (San Diego, Calif.) following procedures recommended by the manufacturer. The ribosomal large subunit protein L32 and glyceraldehyde-3-phosphate dehydrogenase GAPDH signals were used to normalize Cyclin expression at the different time points during CCl₄ liver regeneration. Antisense RNA probes for mouse Cdc25a and Cdc25b were generated from Atlas cDNA plasmids purchased from Clontech (Paolo Alto, Calif.).

RNase protection assays were performed in duplicate to examine the temporal expression patterns of the Cyclin genes in CCl₄ regenerating TG and WT livers. Compared with regenerating WT liver, regenerating TG liver displayed early increases in expression of S-phase promoting Cyclin D1 and E genes between 24 to 36 hours after CCl₄ injury, corresponding to the G1/S transition of the cell cycle. The CCl₄ regenerating TG livers displayed a more significant peak in CyclinD1 expression compared with the regenerating WT livers (FIG. 13A), suggesting that premature FoxM1B can induce Cyclin D1 expression and accelerate hepatocyte entry into S-phase. The induction peaks of Cyclin D1 and Cyclin E expression following CCl₄ liver injury in TG mice differ from those observed in the PHx liver regeneration model. Regenerating TG liver displayed a persistent increase in hepatic Cyclin D1 levels from 28 hours post PHx until initiation of DNA replication, and no changes were found in the induction of Cyclin E expression (Ye et al., 1999, Mol. Cell Biol. 19: 8570-8580). Regenerating livers induced by PHx or CCl₄ both exhibit early activation of Cyclin A2 expression (FIG. 13D, Id.). Cyclin A2 complexes with CDK2 and is essential for S-phase progression by mediating E2F phosphorylation, which inactivates its DNA binding activity (Dynlacht et al, 1994, Genes Dev. 8: 1772-1786; Xu et al., 1994, Mol. Cell Biol. 14: 8420-8431).

As observed in previous PHx regeneration studies, which demonstrated an 8 hour acceleration in entry into mitosis coinciding with early expression of Cyclin B1 and B2 genes (Ye et al., 1999, Mol Cell Biol. 19: 8570-8580), CCl₄-regenerating TG liver displayed early hepatic expression of Cyclin B1 and B2 genes (FIG. 13C). Also, both liver regeneration models displayed early induction of Cyclin F levels at the peak of hepatocyte DNA replication (FIG. 13E). Cyclin F may mediate nuclear localization of the Cyclin B proteins and entry into mitosis (Kong et al., 2000, EMBO J 19: 1378-1388). The present results suggest that early Cyclin F expression may elicit earlier TG hepatocyte entry into M-phase by facilitating Cyclin B nuclear localization. In addition, analysis of these liver regeneration models studies suggest that FoxM1B activates distinct S-phase promoting pathways following CCl₄ liver injury, but they displayed activation of similar Cyclin genes for accelerated entry into M-phase.

RNase protection assays also demonstrated that high levels of Cdc25a mRNA are maintained between 24 and 40 hours after CCl₄ injury in regenerating TG liver, while Cdc25a expression in regenerating WT liver decreases sharply after the 28 hour time point (FIGS. 13F and G). Cdc25a expression was sustained through the peak of TG hepatocyte DNA replication allowing for progression into S-phase through activation of the CyclinD1/CDK4 complex. At the peak of TG hepatocyte replication, an increase in Cdc25b (cdc25M2) phosphatase levels was observed (FIG. 13G). Early activation of Cdc25b mRNA levels was seen in regenerating TG liver at 36 hours post CCl₄ injury, whereas its expression did not increase in WT regenerating liver until the 40 hour time point (FIG. 13G). Cdc25b regulated M-phase progression by activating the mitotic kinase Cdk1/cyclin B via dephosphorylation (Nilsson et al., 2000, Prog. Cell Cycle Res. 4: 107-114; Sebastian et al., 1993, Proc. Natl. Acad. Sci. USA. 90: 3521-3524; Trembley et al., 1996, Cell Growth Differ. 7: 903-916). Early expression of Cdc25b promotes entry into mitosis by activating cdk1-cyclinB kinase activity, which is required to initiate and execute mitosis (division of duplicated chromosomes to daughter cells).

### Example 8

Expression of FoxM1B by Adenoviral Delivery of the FoxM1B Gene to Livers of Mice

Twelve month old Balb/c mice were obtained from the National Institute of Aging and were infected by tail vein injection with either adenovirus vectors expressing FoxM1B (AdFoxM1B) or adenovirus as a control (AdCon) (1×10¹¹ purified adenovirus particles). The adenovirus expressing FoxM1B (AdFoxM1B) was generated by subcloning the 2.7 kB EcoRI-HindIII fragment of the human FoxM1B cDNA into the adenovirus shuttle vector pGEMCMV NEW (gift from J. R. Nevins, Duke University). Greater than 95% of the adenovirus infects the liver after tail vein injection with minimal infection of other organs. Adenovirus is efficiently delivered to most cells throughout the liver parenchyma. Mouse tail vein injection of AdFoxM1B effectively increases in vivo hepatic expression of FoxM1B. Two days after tail vein injection, infected mice subjected to partial hepatectomy (PHx) operation as described above. PHx operation was performed two days after adenovirus infection to avoid the initial acute phase response to viral infection, which is completed within the first 36 hours following adenovirus infection. An intraperitoneal (IP) injection of a phosphate buffered saline (PBS) solution containing 10 mg/mL BrdU (Sigma; 50 μg/g body weight) was administered two hours prior to harvesting the remnant regenerating liver, which were harvested at different intervals between 24 and 48 hours following surgery as previously described (Ye et al., 1999, Mol. Cell Biol. 19:8570-8580).

The liver tissue was used to prepare total RNA or paraffin embedded for immunohistochemical staining of BrdU incorporation into DNA to monitor hepatocyte DNA replication as described previously. RNase protection assays were performed with the FoxM1B RNase protection probe as described above, and demonstrated that AdFoxM1B infection elicited a large increase in FoxM1B mRNA (FIG. 14A). For comparison, RNase protection assays were performed on liver RNA isolated from regenerating livers of 2 month-old (young) mice. Significant increases in FoxM1B expression were observed in these samples between 36 and 44 hours following PHx high expression levels and were sustained for the duration of the liver regeneration experiment (FIG. 14A). In contrast, RNase protection assays with RNA from regenerating livers of old-aged mice that were AdCon infected displayed only minimal increase in FoxM1B mRNA at 24 hours post PHx with a second increase at 40 hours (FIG. 14A). Also, a small increase in FoxM1B expression was observed throughout the time points examined from uninfected regenerating liver of old mice (FIG. 14A).

Paraffin embedded liver tissues were subjected to immunostaining with anti-BrdU antibodies and the expression pattern of the FoxM1B protein was examined by immunohistochemistry using FoxM1B protein as described above. The adenovirus mediated increase in FoxM1B expression stimulated an earlier peak in hepatocyte DNA replication at 32 hours post PHx (FIG. 14B), which normally occurs at 40 hours post PHx in young Balb/c mice. Consistent with the role of FoxM1B in mediating progression into S-phase, regenerating liver infected with AdCon or mock infected lacked significant increase in hepatocyte DNA replication (FIG. 14B). Hepatocyte mitotic figures were examined and are represented graphically in FIG. 14C. Adenovirus mediated increase in FoxM1B expression stimulated hepatocyte mitosis between 36 to 44 hours post PHx compared to regenerating livers of old mice infected with either control adenovirus or uninfected (FIG. 14C). Immunohistochemical staining of regenerating liver from old mice infected with AdCon exhibited undetectable nuclear protein levels of FoxM1B following PHx (FIG. 15, left panel). Nuclear FoxM1B protein expression was observed in all time points between 24 and 36 hours (FIG. 15, right panel).

These results show the adenovirus mediated increase in hepatic levels of FoxM1B restored hepatocyte progression into S-phase and mitosis at a rate similar to that found in young regenerating liver.

### Example 9

Expression of Cell Cycle Regulatory Genes is Restored in Regenerating Livers of Old-Aged Mice Expressing AdFoxM1B

Expression of S-Phase Promoting Gene

To identify cell cycle regulatory genes whose expression is restored in regenerating liver of old mice infected with AdFoxM1B, RNase protection assays were performed as described in duplicate with probes against various Cyclin genes with RNA isolated from regenerating liver of old-aged Balb/c mice infected with Adcon or AdFoxM1B as above.

Increased FoxM1B expression caused by infection of AdFoxM1B in old-aged regenerating liver was associated with elevated expression of the S-phase promoting Cyclin D1 gene at 28 hours post PHx (FIG. 16D). Likewise, Cyclin E displayed a significant increase between 28 and 32 hours post PHx in old mice infected with AdFoxM1B (FIG. 16F). Consistent with diminished hepatocyte entry into S-phase, regenerating liver of old mice infected with AdCon displayed significant decreases in Cyclin D1 and Cyclin E expression during the G1/S transition of the cell cycle (FIG. 16D and F). Elevated FoxM1B levels also restored increased expression of Cyclin A2 in regenerating liver of old mice infected with AdFoxM1B (FIG. 16A).

Taken together, these data indicate that restoring FoxM1B expression in regenerating liver of old mice stimulates induction of S-phase promoting Cyclin D1, Cyclin E and Cyclin A2, which served to facilitate hepatocyte entry and progression through S-phase.

Expression of M-Phase Promoting Genes

RNase protection assays were performed with probes against Cyclins involved in M-phase progression as described. At the peak of hepatocyte DNA replication (24 to 32 hour post PHx), only regenerating liver from old mice infected with AdFoxM1B displayed significant induction of Cyclin B1 and Cyclin B2 (FIGS. 16B and C; 24 to 32 hours post PHx). Concomitant with induction of Cyclin B levels, a significant increase in Cyclin F levels was evident in 12-month old regenerating liver infected with AdFoxM1B (FIG. 16G). In addition, elevated levels of Cyclin G were observed during the period of hepatocyte DNA replication (FIG. 16H).

Taken together, these liver regeneration studies indicate that adenovirus increased FoxM1B expression in old mice restores induction of M-phase promoting Cyclin B1, Cyclin B2, Cyclin F, and Cyclin G genes which are required for M-phase progression.

### Example 10

Proliferation and Mitosis in Conditional FoxM1B Knockout Mice During Liver Regeneration

FoxM1B knockout mice die immediately after birth. Therefore, to examine the role of FoxM1B in adult liver regeneration conditional FoxM1B knockout mice were generated using a triple-LoxP FoxM1B targeting vector to create a “Floxed” FoxM1B targeted locus (see FIG. 17 for schematic of vector). Cre recombinase mediated deletion of the FoxM1 genomic sequences spanning the two LoxP sites removes the entire winged helix DNA binding domain and the C-terminal transcriptional activation domain, thereby preventing expression of functional FoxM1 isoforms. Following standard electroporation and culture of mouse embryonic stem (ES) cells to select for homologous recombination (G418 and gangcyclovir), homologous recombinants were identified by Southern blotting of ES cell genomic DNA.

Mouse blastocysts were injected with the ES cells comprising the “Floxed” (fl/+) FoxM1B targeted allele, and chimeric mice with germ line transmission were selected. Viable mice homozygous for the “Floxed” (fl/fl) FoxM1B targeted allele were generated. Mice either homozygous (fl/fl) or heterozygous (fl/+) for the FoxM1B (fl) allele were verified by PCR amplification of mouse genomic DNA with primers that flanked the LoxP site. Breeding the albumin promoter Cre recombinase transgene into the FoxM1B (fl/fl) mouse genetic background allowed hepatocyte deletion of the FoxM1B locus within six weeks after birth, which was verified by Southern blot using liver genomic DNA.

The role of FoxM1B in hepatocyte proliferation was examined by performing liver regeneration studies with FoxM1B fl/fl and FoxM1B −/− mice in which the FoxM1B gene was deleted in hepatocytes by the albumin Cre recombinase transgene. Eight-week old FoxM1B −/− mice were subjected to partial hepatectomy (PHx) and their regenerating livers were harvested at different intervals between 24 and 52 hours following surgery (Wang et al., 2001, Proc. Natl. Acad. Sci. USA 98: 11468-11473). Hepatocyte DNA synthesis was monitored by immunohistochemical staining of 5-bromo-2′-deoxyuridine (BrdU) incorporation into DNA as described above.

The FoxM1B fl/fl mice exhibited an 8-hour earlier expression of FoxM1B (at 32-hrs post PHx) in comparison to regenerating WT liver (Id.). Because FoxM1B is predominantly regulated at the post-transcriptional level, the LoxP neo construct at the 3′ end of the FoxM1B gene is presumably stabilizing its mRNA and thus enhancing induced FoxM1B levels. FoxM1B (fl/fl) mice exhibited a bifunctional S-phase peak in BrdU incorporation post PHx (FIG. 18A), while a significant reduction in DNA replication was observed in FoxM1B (−/−) regenerating livers (FIG. 18A). In addition, progression into mitosis was significantly reduced in regenerating hepatocytes of FoxM1B (−/−) mice as evidenced by the paucity of mitotic figures between 36 to 52 hours post PHx (FIG. 18B).

RNase protection assays were performed in duplicate to identify cell cycle regulatory genes, whose expression is diminished in regenerating liver of FoxM1B −/− mice, (FIG. 19A). Minimal changes in cyclin D or cyclin E mRNA levels in regenerating liver of FoxM1B (−/−) mice were detected (FIG. 19A). However, Western blot analysis revealed elevated p21 protein levels in regenerating FoxM1B −/− hepatocytes compared to the FoxM1B fl/fl equaled controls (FIG. 19B). Since p21 protein inhibits cyclin/cdk activity, increased p21 protein levels provide an explanation for the decreases in DNA replication in regenerating FoxM1B −/− hepatocytes.

Diminished progression into mitosis of regenerating FoxM1B −/− livers is consistent with reduction in Cdc25B mRNA levels between 40 to 48 hour time points following the PHx operation. Western blot analysis with cdk-1 specific phospho-Tyrosine 15 antibodies demonstrated increased cdk-1 phosphorylation in FoxM1B deficient hepatocytes (FIG. 19C), a finding consistent with diminished levels of the Cdc25B phosphatase leading to reduced cdk1 activity (Nilsson et al., 2000, Prog. Cell Cycle Res. 4: 107-114; Sebastian et al., 1993, Proc. Natl. Acad. Sci. USA 90: 3521-3524; Trembley et al., 1996, Cell Growth Differ. 7: 903-916). In support of diminished cdk1 activity, immunoprecipitation-kinase assays demonstrated that protein extracts from regenerating FoxM1B −/− hepatocytes displayed reduced cdk-1-dependent phosphorylation of the histone H1 substrate (FIG. 19C). Also, reduced cyclin A2, cyclin B1 and cdk1 levels were observed in FoxM1B −/−, but their expression was still increased during the cell cycle.

Collectively, these results suggest that FoxM1B regulates an essential activator of M-phase progression (Cdc25B) and mediates diminished p21 expression that facilitates entry into S-phase.

### Example 11

The Effects of Growth Hormone on Expression and Localization of FoxM1B in the Liver

Two-month old WT and TG CD-1 mice were subjected to intraperitoneal (IP) injection of human growth hormone (Somatropin (Norditropin), Novo Nordisk Pharmaceuticals Inc., Princeton, N.J.; 5 μg per gram body weight) in vehicle buffer (2.2 mg glycine, 0.325 mg Disodium Phosphate Dihydrate (Na₂HPO₄, 2H₂O), 0.275 mg Sodium Phosphate Dihydrate (NaH₂PO₄, 2H₂O), and 11 mg Mannitol per mL of solution). Liver tissue was harvested at various time intervals (from 0 to 3 hours) following growth hormone administration. Liver tissue was paraffin embedded used for immunohistochemical staining with the FoxM1B antibody. Immunohistochemical staining demonstrated that human growth hormone induced nuclear staining of FoxM1B protein in WT mice within one half hour of growth hormone administration (FIGS. 20C-D compared to FIGS. 20A-B) and nuclear staining of FoxM1B protein persisted until the 3 hour time point (FIGS. 20E-H). Nuclear staining of the transgenic FoxM1B protein was induced by growth hormone between 30 minutes and 3 hours following IP administration to the TTR-FoxM1B transgenic mice (FIG. 21). No hepatic nuclear FoxM1B staining was found mouse WT and TG mouse controls injected with the growth hormone vehicle buffer (FIGS. 20-21 panels A and B). These studies demonstrate that growth hormone alone is capable of inducing nuclear localization of FoxM1B protein without liver injury caused by PHx or CCl₄.

Reduced FoxM1B levels are found in regenerating liver of old Balb/c mice (12 month old) compared with young Balb/c mice (2 month old) (FIG. 22). The effect of growth hormone on hepatocyte proliferation and FoxM1B expression in old-aged mice was examined by administering growth hormone to 12 month-old Balb/c mice before and after partial hepatectomy (PHx). Human growth hormone (HGH) or phosphate buffered saline (PBS) was administered to old-aged (12 month-old) Balb/c mice by intraperitoneal (IP) injection (5 μg per gram body weight) one hour before PHx operation. The mice were also given IP injections of HGH or PBS every eight hours after the operations until the regenerating livers were harvested.

Mice were injected with BrdU as described above and their livers were harvested at various time intervals between 24 and 48 hours post-PHx. Portions of the liver tissues were used to prepare total RNA for RNase protection assays. Liver tissues were processed and liver sections were stained with anti-BrdU antibodies as described above. BrdU-stained hepatocytes and visible mitotic figures were counted as previously described (Wang et al, 2001, Proc. Natl. Acad. Sci. U.S.A. 98: 11468-11473). Regenerating hepatocyte DNA replication as measured by BrdU incorporation was similar to levels observed in regenerating livers of young (2 month-old) mice (FIG. 23A). Also, mitosis in the regenerating livers of old-aged mice was similar to mitosis in regenerating livers of young mice (FIG. 23B).

FoxM1B expression measured by RNase protection assays was elevated in the regenerating livers of old mice that received periodic HGH injections during the regeneration process (FIG. 22). In addition, HGH treatments restored expression of the FoxM1B target gene Cdc25B phosphatase to levels found in young regenerating livers.

These studies suggest that FoxM1B expression is stimulated by growth hormone in regenerating liver.

### Example 12

Growth Hormone Induces Nuclear Localization of FoxM1B Protein in Quiescent Liver Cells

Green fluorescent protein was fused in frame with FoxM1B amino acids 1 to 748 and the CMV promoter was used to drive the expression of the GFP-FoxM1B fusion protein. The CMV-GFP-FoxM1B expression vector was delivered in 2.5 mL of saline via mouse tail vein injection. The technique has previously demonstrated transduction of DNA expression plasmids in 10% of hepatocytes in vivo. Livers from one group of transduced animals were harvested and processed as described above. A second group of mice transduced with the CMV-GFP-FoxM1B expression vector were given IP injections of HGH 45 minutes before their livers were harvested. Liver sections from both groups were examined under fluorescent microscope. GFP-FoxM1B resided in the cytoplasm of quiescent hepatocytes from animals not treated with HGH (FIG. 24C) while GFP-FoxM1B displayed nuclear localization in hepatocytes from the second group of mice (FIG. 24D). As a control, a third group of mice were transduced with CMV-GFP-FoxM1B-NLS (NLS =SV40 Large T-antigen nuclear localization sequence) (FIG. 24B). The pattern of nuclear localization of GFP-FoxM1B induced by HGH was similar to localization of the dysregulated GFP-FoxM1B-NLS. These results demonstrated that growth hormone was sufficient to induce nuclear localization of FoxM1B protein in quiescent hepatocytes.

It should be understood that the foregoing disclosure emphasizes certain specific embodiments of the invention and that all modifications or alternatives equivalent thereto are within the spirit and scope of the invention as set forth in the appended claims.

We claim:

1. A method of inducing transient nuclear localization of FoxM1B protein in a mammalian liver cell that expresses FoxM1B protein, comprising the step of contacting the liver cell with growth hormone for a time and at a concentration sufficient to have a growth stimulating effect.

2. The method of claim 1, wherein the growth hormone is human growth hormone.

3. The method of claim 1, wherein the mammalian liver cell comprises a recombinant nucleic acid construct comprising a nucleic acid sequence that encodes a protein as set forth in SEQ ID NO: 2 operatively linked to a control sequence, whereby the liver cell expresses FoxM1B protein.

4. The method of claim 3, wherein the control sequence is a liver-specific promoter sequence.

5. The method of claim 4, wherein the liver-specific promoter is a promoter from human α1-antitrypsin, mouse α1-antitrypsin, albumin promoter, serum amyloid A, transthyretin, or hepatocyte nuclear factor 6.

6. The method of claim 5, wherein the liver-specific promoter is induced by growth hormone.

7. The method of claim 3, wherein the recombinant nucleic acid construct is a vector.

8. The method of claim 7, wherein the recombinant nucleic acid construct is a viral vector.

9. The method of claim 8, wherein the viral vector is an adenovirus vector, an adeno-associated virus vector, a retrovirus vector, herpes simplex virus vector, or vaccinia virus vector.

10. The method of claim 3, wherein the recombinant nucleic acid construct is introduced into the mammalian liver cell within a liposome.

11. The method of claim 3, wherein the recombinant nucleic acid construct is introduced into the mammalian liver cell in vivo, comprising the step of administering to a mammal a vector carrying the nucleic acid molecule operatively linked to a control sequence.

12. The method of claim 11, wherein the mammal has liver damage.

13. The method of claim 12, wherein the liver damage is associated with a liver disease.

14. The method of claim 13, wherein the disease is cirrhosis, biliary atrisia, hepatitis B, or hepatitis C.

15. The method of claim 12, wherein the liver damage occurs from microbial infection or exposure to chemical or environmental toxins.

16. The method of claim 15, wherein the liver damage occurs from exposure to viruses, parasites, alcohol, tobacco, acetaminophen, inhalation anesthetics, aflotoxin, allyl alcohol, carbon tetrachloride, or any combination thereof.

17. A method of treating a mammal having liver damage comprising the step of contacting the liver with an amount of growth hormone sufficient to cause nuclear localization of FoxM1B protein.

18. The method of claim 17, wherein the mammal is a human.

19. The method of claim 17, wherein the growth hormone is human growth hormone.

20. The method of claim 17, wherein the liver comprises a recombinant nucleic acid construct comprising a nucleotide sequence that encodes a protein as set forth in SEQ ID NO: 2 operatively linked to a control sequence, wherein cells of the liver produce FoxM1B protein thereby.

21. The method of claim 20, wherein the recombinant nucleic acid construct is a vector.

22. The method of claim 21, wherein the recombinant nucleic acid construct is a viral vector.

23. The method of claim 22, wherein the viral vector is an adenovirus vector, an adeno-associated virus vector, a retrovirus vector, herpes simplex virus vector, or vaccinia virus vector.

24. The method of claim 20, wherein the recombinant nucleic acid construct is delivered to the mammalian cell within a liposome.

25. The method of claim 20, wherein the control sequence is a liver-specific promoter sequence.

26. The method of claim 25, wherein the liver-specific promoter is human α1-antitrypsin, mouse α1-antitrypsin, albumin promoter, serum amyloid A, transthyretin, or hepatocyte nuclear factor 6.

27. The method of claim 26, wherein the liver-specific promoter is induced by growth hormone.

28. The method of claim 17, wherein the liver damage is associated with a liver disease.

29. The method of claim 28, wherein the liver disease is cirrhosis, biliary atrisia, hepatitis B, or hepatitis C.

30. The method of claim 17 wherein the liver damage occurs from microbial infection or exposure to chemical or environmental toxins.

31. The method of claim 30, wherein the liver damage occurs from exposure to a virus, a parasite, alcohol, tobacco, acetaminophen, inhalation anesthetics, aflotoxin, allyl alcohol, carbon tetrachloride, or any combination thereof.

32. A method of stimulating liver regeneration in a mammal, comprising the step of contacting liver cells in the mammal with growth hormone, wherein the liver cells express FoxM1B protein.

33. The method of claim 32, wherein the liver cells comprise a recombinant nucleic acid construct comprising a nucleotide sequence that encodes a protein as set forth in SEQ ID NO: 2 operatively linked to a control sequence into the liver cells, whereby the liver cells express FoxM1B protein.

34. The method of claim 33, wherein the control sequence is a liver-specific promoter sequence.

35. The method of claim 34, wherein the liver-specific promoter is human α1-antitrypsin, mouse α1-antitrypsin, albumin promoter, serum amyloid A, transthyretin, or hepatocyte nuclear factor 6.

36. The method of claim 35, wherein the liver-specific promoter is induced by growth hormone.

37. The method of claim 33, wherein the recombinant nucleic acid construct is a vector.

38. The method of claim 37, wherein the recombinant nucleic acid construct is a viral vector.

39. The method of claim 38, wherein the viral vector is an adenovirus vector, an adeno-associated virus vector, a retrovirus vector, herpes simplex virus vector, or vaccinia virus vector.

40. The method of claim 33, wherein the recombinant nucleic acid construct is delivered to the mammalian cell within a liposome.

41. The method of claim 32, wherein the mammal is a human.

42. A method of stimulating liver regeneration comprising the steps of:
a. isolating liver cells from a first mammal;
b. introducing a recombinant nucleic acid construct comprising a nucleotide sequence that encodes a protein as set forth in SEQ ID NO: 2 operatively linked to a promoter sequence into the liver cells, whereby the liver cells express FoxM1B protein;
c. introducing the liver cells that express FoxM1B protein into a second mammal; and
d. administering to the second mammal an amount of human growth hormone sufficient to induce nuclear localization of the FoxM1B protein in the liver cells.

43. The method of claim 42, wherein the liver cells expressing FoxM1B protein are reintroduced into first mammal, and the first mammal is treated with an amount of human growth hormone sufficient to induce nuclear localization of the FoxM1B protein in the liver cells.

44. The method of claim 42, wherein the control sequence is a liver-specific promoter sequence.

45. The method of claim 44, wherein the liver-specific promoter is human α1-antitrypsin, mouse α1-antitrypsin, albumin promoter, serum amyloid A, transthyretin, or hepatocyte nuclear factor 6.

46. The method of claim 45, wherein the liver-specific promoter is induced by growth hormone.

47. The method of claim 42, wherein the recombinant nucleic acid construct is a vector.

48. The method of claim 47, wherein the recombinant nucleic acid construct is a viral vector.

49. The method of claim 48, wherein the viral vector is an adenovirus vector, an adeno-associated virus vector, a retrovirus vector, herpes simplex virus vector, or vaccinia virus vector.

50. The method of claim 42, wherein the recombinant nucleic acid construct is delivered to the mammalian cell within a liposome.

51. The method of claim 42, wherein the first mammal is a human and wherein the second mammal is a human.

52. A method of preventing or ameliorating liver damage in a mammal comprising the step of contacting liver c ells of the mammal with growth hormone, wherein the liver cells express FoxM1B protein.

53. The method of claim 52, wherein the mammal is a human.

54. The method of claim 52, wherein the growth hormone is human g row th hormone.

55. The method of claim 52, wherein the liver cells comprise a recombinant nucleic acid construct comprising a nucleotide sequence that encodes a protein as set forth in SEQ ID NO: 2 operatively linked to a control sequence, whereby the liver cells express FoxM1B protein.

56. The method of claim 55, wherein the recombinant nucleic acid construct is a vector.

57. The method of claim 56, wherein the recombinant nucleic acid construct is a viral vector.

58. The method of claim 57, wherein the viral vector is an adenovirus vector, an adeno-associated virus vector, a retrovirus vector, herpes simplex virus vector, or vaccinia virus vector.

59. The method of claim 55, wherein the recombinant nucleic acid construct is delivered to the mammalian cell within a liposome.

60. The method of claim 55, wherein the control sequence is a liver-specific promoter sequence.

61. The method of claim 60, wherein the liver-specific promoter is human α1-antitrypsin, mouse α1-antitrypsin, albumin promoter, serum amyloid A, transthyretin, or hepatocyte nuclear factor 6.

62. The method of claim 61, wherein the liver-specific promoter is induced by growth hormone.

63. The method of claim 52, wherein the liver damage is associated with a liver disease.

64. The method of claim 63, wherein the liver disease is cirrhosis, biliary atrisia, hepatitis B, or hepatitis C.

65. The method of claim 52, wherein the liver damage occurs from microbial infection or exposure to chemical or environmental toxins.

66. The method of claim 65, wherein the liver damage occurs from exposure to a virus, a parasite, alcohol, tobacco, acetaminophen, inhalation anesthetics, aflotoxin, allyl alcohol, carbon tetrachloride, or any combination thereof.

67. A method of preventing or ameliorating liver damage in a patient comprising the steps of:
a. introducing into the patient liver cells having a recombinant nucleic acid construct comprising a nucleotide sequence that encodes a protein as set forth in SEQ ID NO: 2 operatively linked to a control sequence, whereby the liver cells express FoxM1B protein; and
b. treating the patient with an amount of growth hormone sufficient to induce nuclear localization of FoxM1B protein.

68. The method of claim 67, wherein the patient is a human.

69. The method of claim 67, wherein the control sequence is a liver-specific promoter sequence.

70. The method of claim 69, wherein the liver-specific promoter is human α1-antitrypsin, mouse α1-antitrypsin, albumin promoter, serum amyloid A, transthyretin, or hepatocyte nuclear factor 6.

71. The method of claim 70, wherein the liver-specific promoter is induced by growth hormone.

72. The method of claim 67, wherein the recombinant nucleic acid construct is a vector.

73. The method of claim 72, wherein the recombinant nucleic acid construct is a viral vector.

74. The method of claim 73, wherein the viral vector is an adenovirus vector, an adeno-associated virus vector, a retrovirus vector, herpes simplex virus vector, or vaccinia virus vector.

75. The vector of claim 67, wherein the recombinant nucleic acid construct is delivered to the mammalian cell within a liposome.

76. A method of preventing or ameliorating liver damage in a liver to be transplanted into a recipient comprising the steps of:
a. surgically removing all or a portion of a liver from a donor; and
b. contacting the liver with an amount of growth hormone sufficient to induce nuclear localization of FoxM1B protein.

77. The method of claim 76, wherein the recipient is a mammal and the donor is a mammal.

78. The method of claim 77, wherein the recipient is a human and the donor is a human.

79. The method of claim 78, wherein the growth hormone is human growth hormone.

80. The method of claim 76, wherein prior to surgically removing all or a portion of the liver from the donor, the donor is treated with an amount of growth hormone sufficient to induce nuclear localization of FoxM1B protein in the liver, and after all or a portion of the liver is removed from the donor, the donor is treated with an amount of growth hormone sufficient to induce nuclear localization of FoxM1B protein in the liver.

81. The method of claim 80, wherein the growth hormone is human growth hormone.

82. The method of claim 76, wherein prior to surgically removing all or a portion of the liver from the donor, the donor is treated with an amount of growth hormone sufficient to induce expression and nuclear localization of FoxM1B protein in the liver, and after all or a portion of the liver is removed from the donor, the donor is treated with an amount of growth hormone sufficient to induce expression and nuclear localization of FoxM1B protein in the liver.

83. The method of claim 82, wherein the growth hormone is human growth hormone.

84. The method of claim 76, wherein the liver comprises a recombinant nucleic acid construct comprising a nucleotide sequence that encodes a protein as set forth in SEQ ID NO: 2 operatively linked to a control sequence, whereby the liver expresses FoxM1B protein.

85. The method of claim 84, wherein the control sequence is a liver-specific promoter sequence.

86. The method of claim 85, wherein the liver--specific promoter is human α1-antitrypsin, mouse α1-antitrypsin, albumin promoter, serum amyloid A, transthyretin, or hepatocyte nuclear factor 6.

87. The method of claim 86, wherein the liver-specific promoter is induced by growth hormone.

88. The method of claim 84, wherein the recombinant nucleic acid construct is a vector.

89. The method of claim 88, wherein the recombinant nucleic acid construct is a viral vector.

90. The method of claim 89, wherein the viral vector is an adenovirus vector, an adeno-associated virus vector, a retrovirus vector, herpes simplex virus vector, or vaccinia virus vector.

91. The vector of claim 84, wherein the recombinant nucleic acid construct is delivered to the mammalian cell within a liposome.

92. A method of screening for compounds that induce expression of FoxM1B in mammalian cells, wherein the FoxM1B protein can be translocated into the nucleus, comprising the steps of:
a. contacting a plurality of cells that comprise the FoxM1B gene, wherein the FoxM1B protein is not expressed under conventional culture conditions, with a candidate compound in the presence of growth hormone;
b. contacting a plurality of cells that comprise the FoxM1B gene, wherein the FoxM1B protein is not expressed under normal culture conditions, with the candidate compound in the absence of growth hormone; and
c. assaying FoxM1B expression and localization in the cells from step (a) and step (b);
wherein a candidate compound is identified if FoxM1B is localized in the nuclei of cells from step (a) and in the cytoplasm of cells from step (b).

93. The method of claim 92, wherein the liver cells comprise a recombinant nucleic acid construct comprising a nucleotide sequence that encodes a protein as set forth in SEQ ID NO: 2 operatively linked to a control sequence, whereby the liver cells express FoxM1B protein.

94. The method of claim 93, wherein the control sequence is a liver-specific promoter sequence.

95. The method of claim 94, wherein the liver-specific promoter is human α1-antitrypsin, mouse α1-antitrypsin, albumin promoter, serum amyloid A, transthyretin, or hepatocyte nuclear factor 6.

96. The method of claim 95, wherein the liver-specific promoter is induced by growth hormone.

97. The method of claim 93, wherein the recombinant nucleic acid construct is a vector.

98. The method of claim 97, wherein the recombinant nucleic acid construct is a viral vector.

99. The method of claim 98, wherein the viral vector is an adenovirus vector, an adeno-associated virus vector, a retrovirus vector, herpes simplex virus vector, or vaccinia virus vector.

100. The vector of claim 93, wherein the recombinant nucleic acid construct is delivered to the mammalian cell within a liposome.

101. A pharmaceutical composition comprising the compound selected in claim 92.

102. A method of treating a mammal having liver damage, comprising administering to the mammal an amount of the pharmaceutical composition of claim 101 in combination with growth hormone.

103. The method of claim 102, wherein the liver damage is caused by a liver disease.

104. The method of claim 103, wherein the liver disease is cirrhosis, biliary atrisia, hepatitis B, or hepatitis C.

105. The method of claim 102, wherein the liver damage occurs from microbial infection or exposure to chemical or environmental toxins.

106. The method of claim 105, wherein the liver damage occurs from exposure to a virus, a parasite, alcohol, tobacco, acetaminophen, inhalation anesthetics, aflotoxin, allyl alcohol, carbon tetrachloride, or a combination thereof.

107. The method of claim 102, wherein the mammal is a human.

108. The method of claim 102, wherein the growth hormone is human growth hormone.

109. A method of screening for compounds that induce nuclear localization of FoxM1B protein, comprising the steps of:
a. contacting a cell with a candidate compound, wherein the cell expresses FoxM1B protein; and
b. examining localization of FoxM1B protein in the cell;
wherein the candidate compound is identified if FoxM1B protein is localized in the nucleus of the cell.

110. The method of claim 109, wherein the cell comprises a recombinant nucleic acid construct comprising a nucleotide sequence that encodes a protein as set forth in SEQ ID NO: 2 operatively linked to a control sequence, whereby the cell expresses FoxM1B protein.

111. The method of claim 110, wherein the control sequence is a liver-specific promoter sequence.

112. The method of claim 111, wherein the liver-specific promoter is human α1-antitrypsin, mouse α1-antitrypsin, albumin promoter, serum amyloid A, transthyretin, or hepatocyte nuclear factor 6.

113. The method of claim 112, wherein the liver-specific promoter is induced by growth hormone.

114. The method of claim 110, wherein the recombinant nucleic acid construct is a vector.

115. The method of claim 114, wherein the recombinant nucleic acid construct is a viral vector.

116. The method of claim 115, wherein the viral vector is an adenovirus vector, an adeno-associated virus vector, a retrovirus vector, herpes simplex virus vector, or vaccinia virus vector.

117. The vector of claim 110, wherein the recombinant nucleic acid construct is delivered to the mammalian cell within a liposome.

118. A method of inducing liver cell proliferation comprising the step of contacting a liver cell with growth hormone, wherein the liver cell expresses FoxM1B protein.

119. The method of claim 118, wherein the growth hormone is human growth hormone.

120. The method of claim 118, wherein the liver cell comprises a recombinant nucleic acid construct comprising a nucleic acid sequence that encodes a protein as set forth in SEQ ID NO: 2 operatively linked to a control sequence, whereby the liver cell expresses FoxM1B protein.

121. The method of claim 120, wherein the control sequence is a liver-specific promoter sequence.

122. The method of claim 121, wherein the liver-specific promoter is human α1-antitrypsin, mouse α1-antitrypsin, albumin promoter, serum amyloid A, transthyretin, or hepatocyte nuclear factor 6.

123. The method of claim 122, wherein the liver-specific promoter is induced by growth hormone.

124. The method of claim 120, wherein the recombinant nucleic acid construct is a vector.

125. The method of claim 124, wherein the recombinant nucleic acid construct is a viral vector.

126. The method of claim 125, wherein the viral vector is an adenovirus vector, an adeno-associated virus vector, a retrovirus vector, herpes simplex virus vector, or vaccinia virus vector.

127. The method of claim 120, wherein the recombinant nucleic acid construct is introduced into the liver cell within a liposome.

128. A method of screening for compounds that induce both expression and nuclear localization of FoxM1B protein comprising the steps of:
a. contacting a plurality of cells that comprise the FoxM1B gene, wherein the cells do not express FoxM1B protein under conventional culture conditions with a candidate compound; and
b. assaying FoxM1B expression and localization in the cells,
wherein a candidate compound is identified when FoxM1B is expressed and localized in the nuclei of cells contacted with the compound but not in cells not contacted with the compound.

129. The method of claim 128, wherein the liver cells comprise a recombinant nucleic acid construct comprising a nucleotide sequence that encodes a protein as set forth in SEQ ID NO: 2 operatively linked to a control sequence, whereby the liver cells express FoxM1B protein.

130. The method of claim 129, wherein the control sequence is a liver-specific promoter sequence.

131. The method of claim 130, wherein the liver-specific promoter is human α1-antitrypsin, mouse α1-antitrypsin, albumin promoter, serum amyloid A, transthyretin, or hepatocyte nuclear factor 6.

132. The method of claim 131, wherein the liver-specific promoter is induced by growth hormone.

133. The method of claim 129, wherein the recombinant nucleic acid construct is a vector.

134. The method of claim 133, wherein the recombinant nucleic acid construct is a viral vector.

135. The method of claim 134, wherein the viral vector is an adenovirus vector, an adeno-associated virus vector, a retrovirus vector, herpes simplex virus vector, or vaccinia virus vector.

136. The vector of claim 129, wherein the recombinant nucleic acid construct is delivered to the mammalian cell within a liposome.

137. A pharmaceutical composition comprising the compound selected in claim 128.

138. A method of treating a mammal having liver damage, comprising administering to the mammal an amount of the pharmaceutical composition of claim 137.

139. The method of claim 138, wherein the liver damage is caused by a liver disease.

140. The method of claim 139, wherein the liver disease is cirrhosis, biliary atrisia, hepatitis B, or hepatitis C.

141. The method of claim 138, wherein the liver damage occurs from microbial infection or exposure to chemical or environmental toxins.

142. The method of claim 141, wherein the liver damage occurs from exposure to a virus, a parasite, alcohol, tobacco, acetaminophen, inhalation anesthetics, aflotoxin, allyl alcohol, carbon tetrachloride, or a combination thereof.

143. The method of claim 138, wherein the mammal is a human.

144. The method of claim 138, wherein the pharmaceutical composition is administered in combination with human growth hormone.
